(19) United States
(12) Patent Application Publication
(10) Pub. No.: US 2019/0147228 A1
(43) Pub. Date: May 16, 2019

(54) SYSTEM AND METHOD FOR HUMAN EMOTION AND IDENTITY DETECTION

(71) Applicant: Aloke Chaudhuri, Victor, NY (US)

(72) Inventor: Aloke Chaudhuri, Victor, NY (US)

(21) Appl. No.: 15/811,511

(22) Filed: Nov. 13, 2017

(51) Int. Cl. G06K 9/00; G06Q 30/02

(52) U.S. Cl. CPC G06K 9/00302; A61B 5/163; G06Q 30/0269; G06Q 30/0201

(57) ABSTRACT

Disclosed is a distributed profile building system, gathering video data, audio data, electronic device identification data, and spatial position data from multiple input devices, performing human emotion and identity detection, and gaze tracking, and forming user profiles. Also disclosed is a method for building user profiles using a distributed profile building system by gathering video data, audio data, electronic device identification data, and spatial position data from multiple input devices, performing human emotion and identity detection, and gaze tracking, and forming user profiles.

### CROSS-REFERENCE TO RELATED APPLICATIONS

None

### STATEMENT REGARDING FEDERALLY SPONSORED RESEARCH OR DEVELOPMENT

None

### THE NAMES OF THE PARTIES TO A JOINT RESEARCH AGREEMENT

None

### BACKGROUND OF THE INVENTION

There is an increased pressure for brick and mortar stores to adapt data analytics as part of their marketing and market research strategy in order to compete with online retail sources and to provide better customer service. Online retailers and website owners, through cookies or other tracking tools, can glean a significant amount of information about visitors and their customers. In many cases online retailers and content providers can gather a significant amount of market data about groups and individuals.

Many retailers have adopted an online shopping presence. They can take advantage of customers who want to shop online, and they can use online tools to gather market research data. However, online tools provide little market research data about customers and visitors to physical stores.

Brick and mortar retailers have a tougher time gathering data about their visitors. Many retailers have some form of loyalty program. These programs often require the customer to present a loyalty card or identifying information to obtain discounts or to obtain program benefits. Many retailers have adopted mobile device applications (“apps”) to gather information about their customers. However, both loyalty programs and apps require that a customer actively participates by presenting a card or activating an app to enable data collection. Furthermore, neither solution is effective in gathering information about visitors or one-off shoppers.

Physical retailers often need to resort to third party market data gathering services such as credit card providers, focus groups, or Wi-Fi hotspot analytics. These solutions might provide group trends but rarely individual information. Furthermore, the information is gathered by a third party and customized information and correlations may be limited.

Current camera or video installations in retail locations are generally for security and crime-prevention purposes. More sophisticated retailers may use video installations to gather information about checkout line waiting times or even certain aisle foot traffic patterns. Such use may limit checkout congestion or provide input of aisle popularity. However, neither provides a customizable solution tailored to individual shoppers and the data gathered provides limited to no individual marketing insight. Current solutions do not provide information regarding a person's emotional response relative to merchandise on store shelves, nor do they provide a way to identify visitor demographics or provide easy solutions to correlate emotional responses to identity information to purchasing information. Such information, commonly available to online retailers, is becoming critical for brick and mortar retailers for merchandising optimization, segmentation, and retargeting strategies.

Further applications that have a need for combining emotional responses and identity information include but are not limited to audience measurement solutions for television programs; advertisement response tracking on mobile devices and other personal electronic or computing device; security screening at border checkpoints, airports, or other sensitive facility access points; police body cameras; or various fraud prevention systems at places like legal gambling establishments.

### BRIEF SUMMARY OF THE INVENTION

Disclosed herein is a distributed system for building a plurality of user profiles comprising: a distributed system for building a plurality of user profiles comprising, a user profile from the plurality of user profiles comprising user profile data; at least one profile building system comprising at least one behavioral response analysis system and the plurality of user profiles; at least one behavior learning system comprising at least one behavior learning processor, at least one video data processor, and at least one audio data processor; at least one data input device comprising a data input device processor and an input data module selected from the group consisting of at least one video input module, at least one audio input module, at least one electronic device identification module, at least one spatial position module, and combinations thereof; and a data communication network comprising the at least one profile building system, the at least one behavior learning system, and the at least one data input device.

Further disclosed is a distributed system for building a plurality of user profiles comprising: a distributed system for building a plurality of user profiles comprising, a user profile from the plurality of user profiles comprising user profile data; at least one profile building system building the user profile comprising at least one behavioral response analysis system providing behavioral response analysis data, and the plurality of user profiles; at least one behavior learning system comprising at least one behavior learning processor, at least one video data processor providing video processor data, and at least one audio data processor providing audio processor data; at least one data input device comprising a data input device processor and data input modules providing data selected from the group consisting of at least one video input module providing video data, at least one audio input module providing audio data, at least one electronic device identification module providing electronic device identification data, at least one spatial position module providing spatial position data, and combinations thereof, and a data communication network providing data communication comprising the profile building system, the behavior learning system, and the at least one data input device.

Further disclosed is a method for building a user profile, the method steps comprising: providing at least one data input device of a plurality of data input devices in at least one fixed space collecting and transmitting video data, audio data, mobile electronic device identification data, and spatial position data of a person from a plurality of persons as the person moves throughout the at least one fixed space; at least one behavior learning system receiving video data, audio data, mobile electronic device identification data, and spatial position data, having at least one video data processor processing video data and at least one audio data processor processing audio data; the at least one behavior learning system transmitting mobile electronic device identification data, spatial position data, video processor data and audio processor data; at least one profile building system receiving mobile electronic device identification data, spatial position data, video processor data, and audio processor data, and building the user profile of the plurality of user profiles; wherein the plurality of user profiles are stored in at least one primary data repository; and wherein the user profile is updated for each person from the plurality of persons moving throughout the at least one fixed space.

### BRIEF DESCRIPTION OF THE DRAWINGS

FIG. 1 is a block diagram overview of an embodiment of a distributed system for building a plurality of user profiles.

FIG. 2A is a block diagram of a second embodiment of a distributed system for building a plurality of user profiles.

FIG. 2B is a block diagram of a third embodiment of a distributed system for building a plurality of user profiles.

FIG. 2C is a block diagram of a fourth embodiment of a distributed system for building a plurality of user profiles.

FIG. 3 is a block diagram of an embodiment of a data input device.

FIG. 4 is a block diagram overview of a behavior learning system.

FIG. 5 is a block diagram of an audio processor.

FIG. 6 is a block diagram of a video processor.

FIG. 7 is a block diagram of a behavior learning system showing an emotion and identity detection system and a gaze tracking module.

FIG. 8 is a block diagram of a behavior learning system showing an emotion and identity detection system, a gaze tracking module, and a facial recognition module.

FIG. 9 is a block diagram depicting an emotion and identity detection system.

FIG. 10 is an alternate embodiment of an emotion and identity detection system.

FIG. 11 is a block diagram of an embodiment of a data input device, known as a core data input device, with components of the behavior learning system are within the data input device.

FIG. 12 is a block diagram of a second embodiment of a data input device, known as a core data input device showing behavior learning system modules.

FIG. 13 is a block diagram of an embodiment of a basic data input device, known as an edge data input device.

FIG. 14A is a block diagram of an embodiment of an electronic device identification module.

FIG. 14B is a block diagram of an embodiment of a spatial position module.

FIG. 15 is a block diagram of an electronic device identification module and spatial position module with a shared component.

FIG. 16 is a block diagram of a gaze tracking module.

FIG. 17 is a block diagram of an embodiment of a distributed system for building a plurality of user profiles with all profile building components on a core device.

FIG. 18 is a block diagram of an embodiment of a distributed system for building a plurality of user profiles with some profile building components on a core device but with natural language processing on the behavior learning system.

FIG. 19 is a block diagram of a behavior learning system.

FIG. 20 is a block diagram of an embodiment of data communication between an employee interface device, data input modules, and a profile building system.

FIG. 21 is a block diagram of profile building system and behavioral response analysis system.

FIG. 22 is a block diagram of profile building system, behavioral response analysis system, and distributed behavior learning system.

FIG. 23 is a block diagram of an embodiment of an audio preprocessor.

FIG. 24 is a block diagram of an embodiment of a facial expression recognition module.

FIG. 25 is a block diagram of an embodiment of a demographic analysis module.

FIG. 26 is a block diagram of an embodiment of a phonetic emotional analysis module.

FIG. 27 is a block diagram of an embodiment of a speech recognition module.

FIG. 28 is a block diagram of an embodiment of a natural language processing module.

FIG. 29 is a block diagram of an embodiment of a facial recognition module.

### DETAILED DESCRIPTION

Before explaining some embodiments of the present invention in detail, it is to be understood that the invention is not limited in its application to the details of any particular embodiment shown or discussed herein since the invention comprises still further embodiments, as described by the granted claims.

The terminology used herein is for the purpose of description and not of limitation. Further, although certain methods are described with reference to certain steps that are presented herein in a certain order, in many instances, these steps may be performed in any order as may be appreciated by one skilled in the art, and the methods are not limited to the particular arrangement of steps disclosed herein.

As utilized herein, the following terms and expressions will be understood as follows:

The terms “a” or “an” are intended to be singular or plural, depending upon the context of use.

The term “building” as used in reference to building a user profile or building the user profile refers to creating, updating, maintaining, storing, and/or deleting, the referenced profile, in whole or in part.

The term “communication” refers to information exchange between at least two devices, systems, modules, or objects, wherein information exchanged is transmitted and/or received by each of the at least two devices.

The expression “machine learning system” refers to computerized systems with the ability to automatically learn and improve from experience without being explicitly programmed. Such systems include but are not limited to artificial neural networks, support vector machines, Bayesian networks, and genetic algorithms. Convolutional neural networks and deep learning neural networks are examples of artificial neural networks.

The expressions “electronic device signal” refers to a mobile phone, tablet, or mobile computing device identification signals or transmissions that include but are not limited to media access control addresses (‘MAC ID’), Bluetooth® signals, other electromagnetic identification signals, or combinations thereof.

The expression “fixed space” refers to any defined or bounded three dimensional space including but not limited to a building or structure, a checkpoint, a retail store, a complex of buildings, a stadium, a park, or outdoor space.

The term “network” refers to a group of two or more computer systems linked together for wired and/or wireless electronic signal transmission and/or communication.

The term “planogram” refers to a visual or digital representation of an item's placement within a fixed space, usually in the form of a diagram or mathematical model. Within the context of a retail store, this includes products, and the placement of retail products on shelves.

The expression “primary data repository” refers to a digital mass data storage system which stores, organizes, and analyzes large amounts of structured or unstructured data, where person profiles and other inventive system data are stored. Within the primary data repository, other data may also be stored, including but not limited to, purchasing system data, market research data, electronic kiosk data, or general research data. The primary data repository may further include information from multiple fixed-space locations and is not limited to information from a single fixed-space.

The expression “secondary data repository” refers to a digital mass data storage system. It includes but is not limited to off-site persona data, external observed location and presence data, public social media data, facial image data, or any information available through Wi-Fi hot-spot market data providers, through geocoding, through public social media searches, or through public image searches.

The invention herein will be better understood by reference to the figures wherein like reference numbers refer to like components.

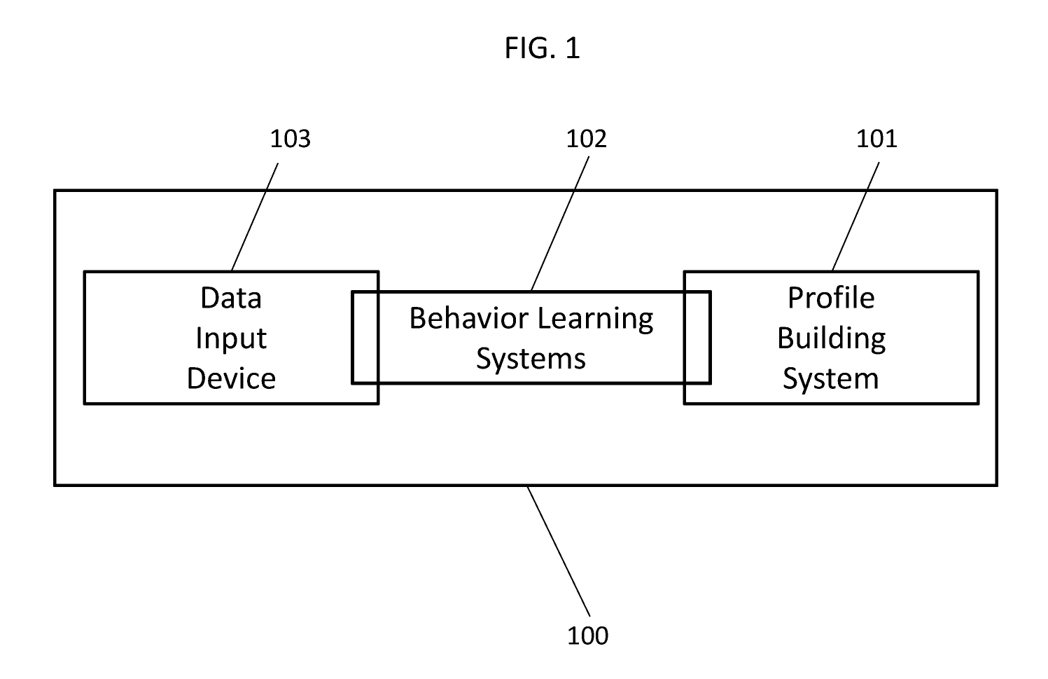

FIG. 1 depicts a block diagram providing an overview of an embodiment of a distributed system for building a plurality of user profiles (100), showing blocks depicting at least one profile building system (101), at least one behavior learning system (102), and at least one data input device (103). The at least one behavior learning system (102) is shown overlapping the at least one profile building system (101) and the at least one data input device (103) to indicate that the at least one behavior learning system (102) may have components within the at least one data input device (103), the at least one behavior learning system (102) may have components within the at least one profile building system (101), or the at least one behavior learning system (102) may have components that are connected but outside the at least one input device (103) and the at least one profile building system (101).

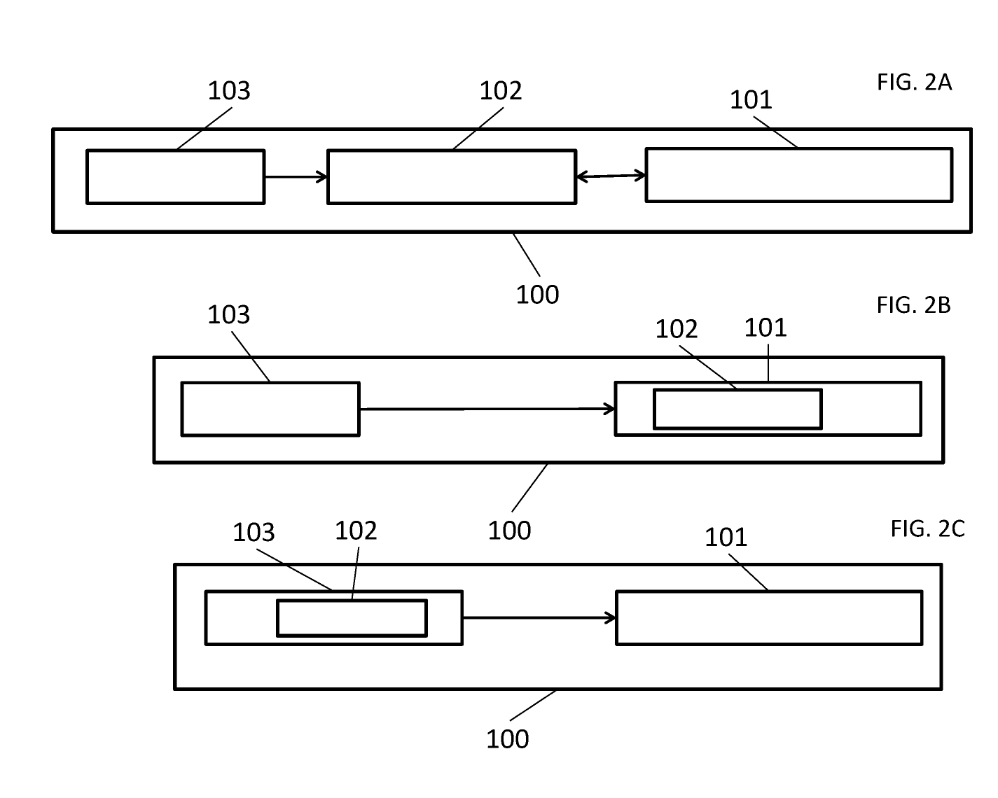

FIG. 2A depicts a block diagram of a distributed system for building a plurality of user profiles (100), where at least one profile building system (101), at least one behavior learning system (102), and at least one data input device (103) are independent systems on independent devices connected to a network.

FIG. 2B depicts a block diagram of a distributed system for building a plurality of user profiles (100), with at least one behavior learning system (102) within at least one profile building system (101), where both are within the same physical computer device or grouping of devices. The at least one behavior learning system (102) and the at least one profile building system (101) are connected to at least one data input device (103) on a network.

FIG. 2C depicts a block diagram of a distributed system for building a plurality of user profiles (100), where at least one behavior learning system (102) is within at least one data input device (103), where both are within the same device or grouping of devices. The at least one behavior learning system (102) and the at least one data input device (103) are connected to at least one profile building system (101) on a network.

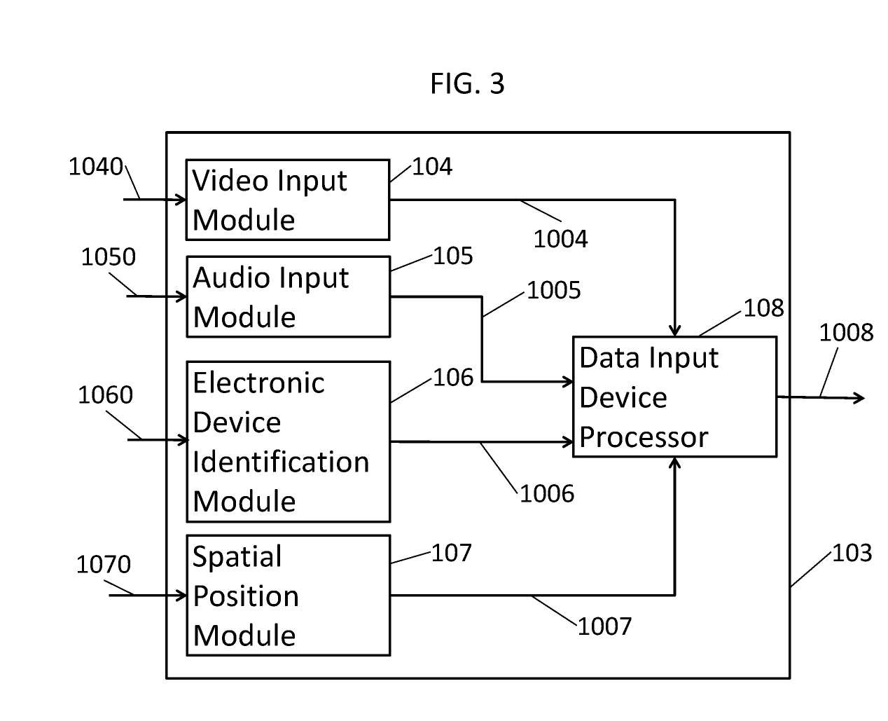

FIG. 3 depicts a block diagram of an embodiment of a data input device (103). Shown are at least one video input module (104), at least one audio input module (105), at least one electronic device identification module (106), and at least one spatial position module (107).

The at least one video input module (104) is shown receiving video input (1040) and providing video data (1004) as output. The at least one audio input module (105) is shown receiving audio input (1050) and providing audio data (1005) as output. The at least one electronic device identification module (106) is shown receiving electronic device signal input (1060) and providing electronic device identification data (1006) as output. The at least one spatial position module (107) is shown receiving spatial position input (1070) and providing spatial position data (1007). Also shown is at least one data input device processor (108), receiving video data (1004), audio data (1005), electronic device identification data (1006), and spatial position data (1007). The at least one data input device processor (108) provides data input device output (1008). The at least one data input device processor (108) may include but is not limited to devices that provide data aggregation, data streaming, data separation, data flow management, data processing, and combinations thereof.

A data input device (103) may also be a distributed device, where components are distributed and may be located in separate physical enclosures in a space or as affixed to an object. A most basic construction may be a simple digital camera with one video input, one audio input, a range finder, and a MAC ID reader. An alternate construction may include a video input, audio input, and MAC ID reader embedded in a consumer electronic device, such as a mobile phone, tablet, or television. A distributed construction example may include: multiple video input modules affixed to shelves surrounding a retail space aisle, audio input modules affixed to shelves at regular intervals, spatial position modules affixed at varying shelf heights and at regular distance intervals along the aisle, a MAC ID reader at the aisle entrance and exit, and all modules connected to a networked multi-processor.

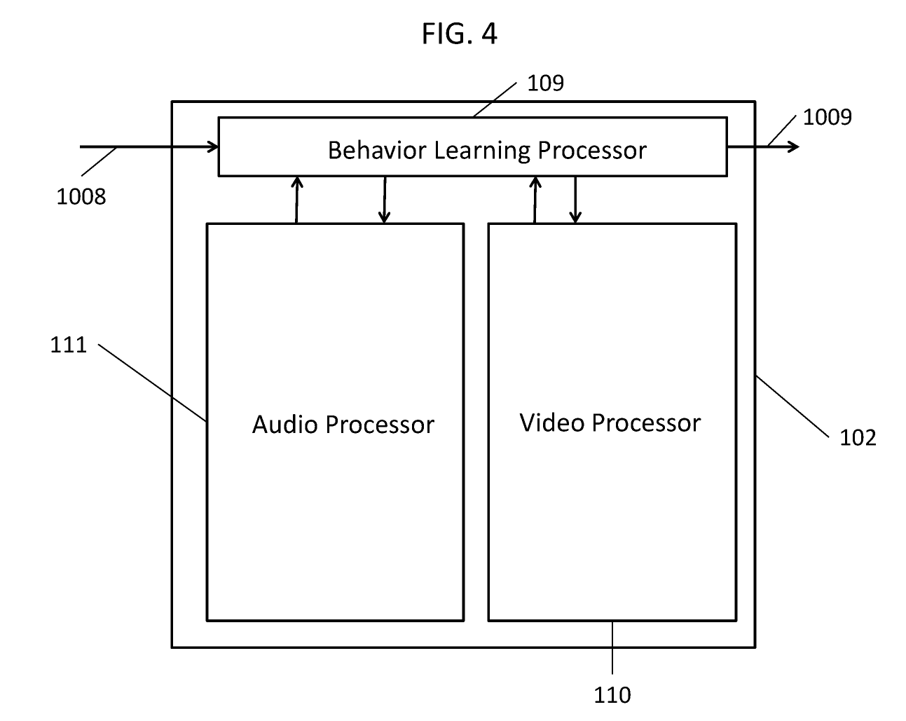

FIG. 4 is a block diagram depicting a broad overview of a behavior learning system (102). Shown are at least one audio processor (111), at least one video processor (110) and at least one behavior learning processor (109). Data input device output (1008) is received by the at least one behavior learning processor (109), communicating with at least one video processor (110) and at least one audio processor (111). The at least one behavior learning processor (109) is shown transmitting behavior learning output data (1009). The behavior learning system may receive data from or transmit data to other system and modules (not shown) and/or the behavior learning system may communicate with other devices or modules (not shown). Data input device output (1008) may be multiple streams of data or a single aggregated stream of data. Behavior learning output data (1009) may be multiple streams of data or a single aggregated stream of data. The at least one behavior learning processor (109) is a form of data processor that may include but is not limited to devices that provide data aggregation, data streaming, data separation, data direction, data communication, and combinations thereof.

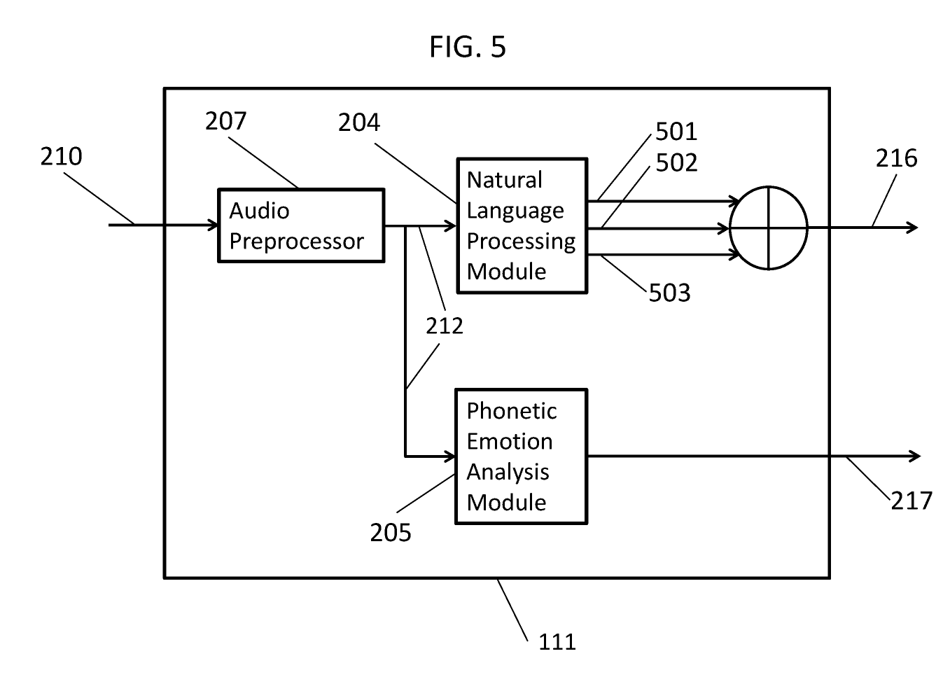

FIG. 5 depicts an audio processor (111) having an audio preprocessor (207), at least one natural language processing module (204), and at least one phonetic emotional analysis module (205). Audio output (210) is received by the audio preprocessor (207), where it is processed. Audio preprocessor output (212) is transmitted to the natural language processing module (204) for further processing and to the phonetic emotional analysis module (205) for further processing. The natural language processing module (204) most commonly provides sentiment data (501), intent data (502), and entity recognition data (503), which is depicted as separate streams but is often combined into a single data stream, natural language output data (216) for transmission. The phonetic emotional analysis module (205) provides phonetic emotional analysis data (217). At least one behavior learning processor (109) may transmit, or aggregate and transmit, the phonetic emotional analysis data (217) and the natural language output (216).

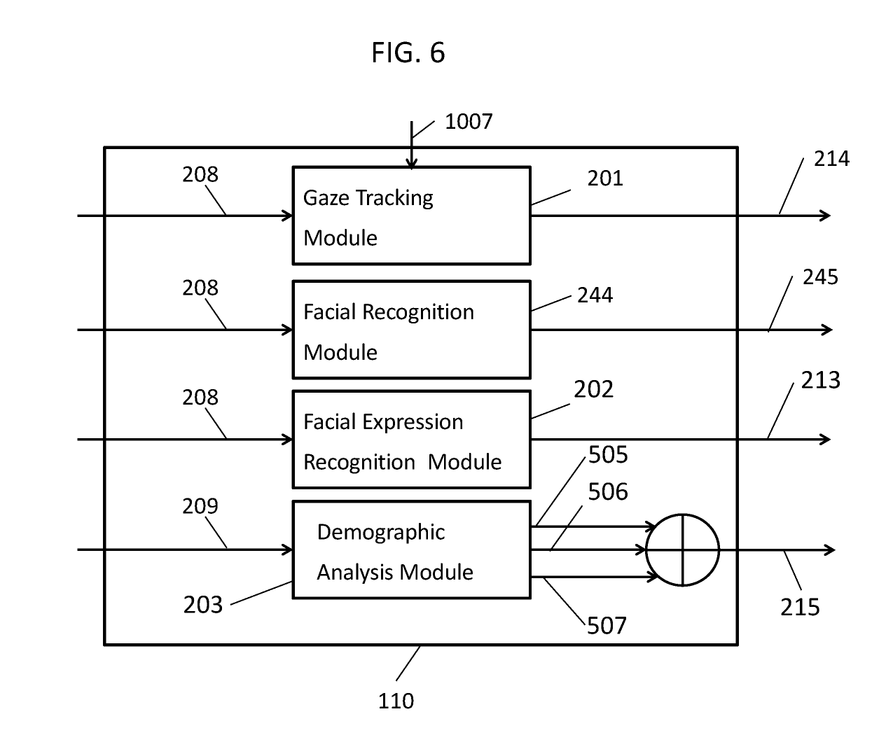

FIG. 6 depicts a video processor (110) having at least one facial expression recognition module (202), at least one gaze tracking module (201), and least one facial recognition module (244), and at least one demographic analysis module (203). In this figure, video output (208) is received by the facial expression recognition module (202), it is processed, and facial expression output data (213) is transmitted. The facial expression output data (213) most commonly comprises facial emotion data. Video output (208) and spatial position data (1007) are shown being received by the gaze tracking module (201), it is processed, and gaze tracking data (214) is transmitted. Video output (208) is shown being received by the facial recognition module (244), it is processed, and facial recognition data (245) is transmitted. Image output data (209) is received and processed by the demographic analysis module (203). The demographic analysis module (203) most commonly transmits age (505), race (506), and gender (507), which is depicted as separate streams but is often combined into a single data stream, demographic analysis data (215).

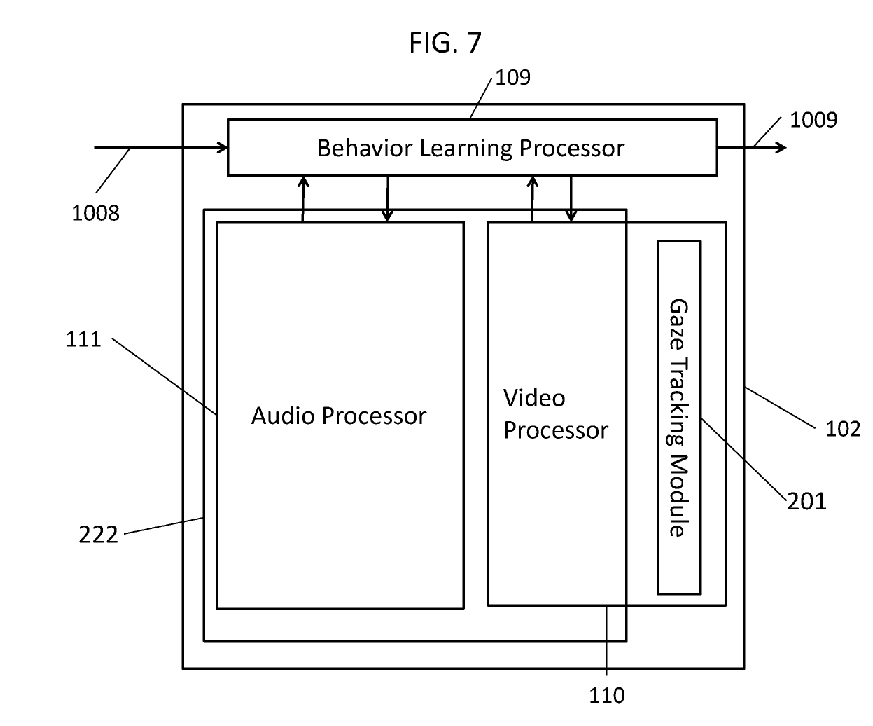

FIG. 7 depicts a behavior learning system (102) showing an emotion and identity detection system (222). An audio processor (111) and a portion of a video processor (110) are shown encapsulated by the emotion and identity detection system (222), with a gaze tracking module (201) being part of the video processor (110) but outside the emotion and identity detection system (222). The emotion and identity detection system (222) refers to a grouping of modules that provide emotion and/or identity data, where the modules may also require at least one machine learning system to provide the emotion and/or identity data. A single machine learning system for all the emotion and identity modules within the audio processor (111) and the video processor (110) may be possible; but it is more likely that there is at least one machine learning system per module within the audio processor (111) and at least one per module within the video processor (110). The gaze tracking module (201) is depicted outside the emotion and identity detection system (222) because its functions are normally performed by an electronic computing device and it normally does not require a machine learning system to perform its functions. While not depicted as part of the emotion and identity detection system, the gaze tracking module (201) may use a machine learning system in certain embodiments to determine a subject's field of view and to identify items viewed by the subject.

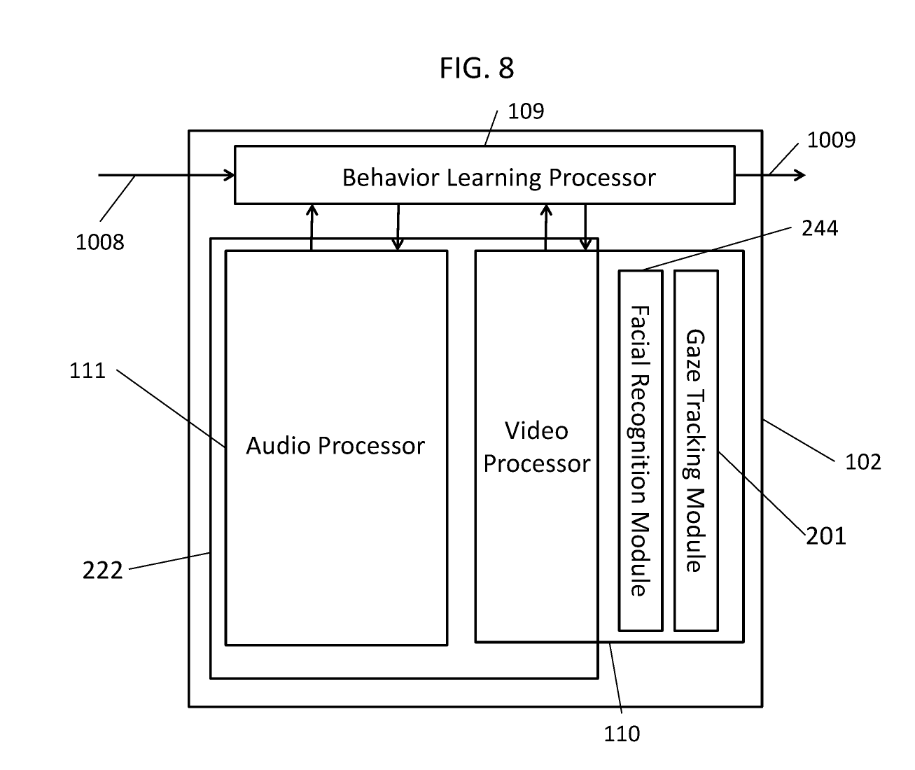

FIG. 8 is similar to FIG. 7 with the difference being that a facial recognition module (244) is depicted outside the emotion and identity detection system (222). The facial recognition module (244) may not always need a machine learning system to perform its functions. In certain embodiments, a gaze tracking module (201) and the facial recognition module (244) may both perform their functions without being a part of the emotion and identity detection system (222).

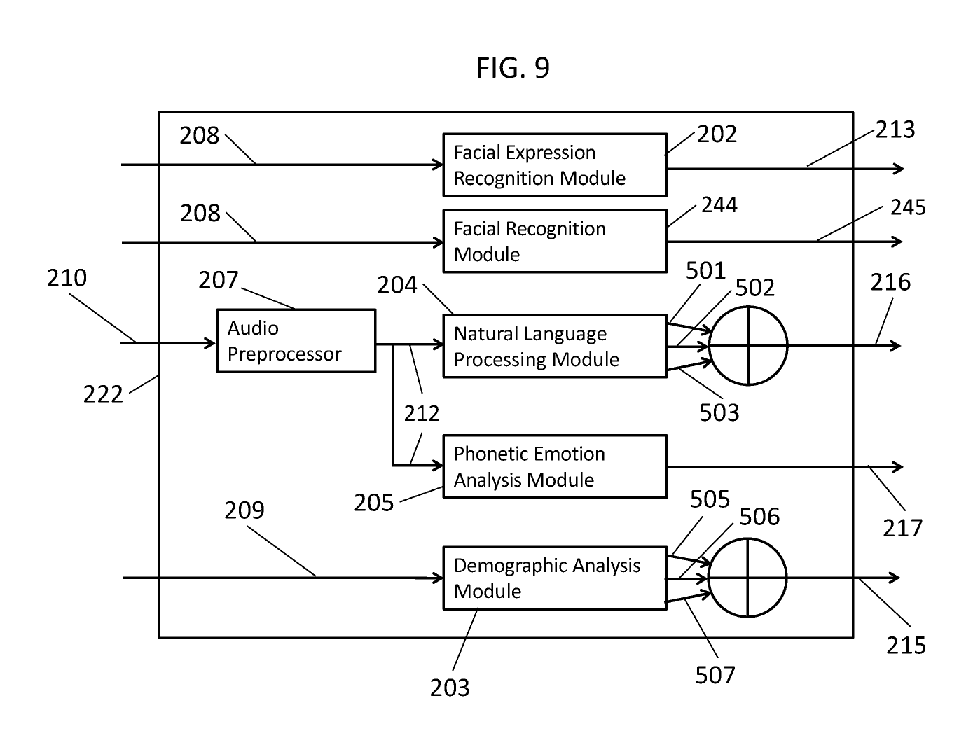

FIG. 9 depicts modules that may be part of the emotion and identity detection system (222). At least one machine learning system, referred to here as an emotion and identity detection system, is needed to perform some of the functions within the behavior learning system. The emotion and identity detection system may encompass multiple machine learning systems. Common embodiments include at least one machine learning system and/or at least one deep learning system. Deep learning systems are a type of machine learning system that generally uses a model based convolutional neural networks with a high level of dimensionality.

Shown are an audio preprocessor (207), a facial expression recognition module (202), a facial recognition module (244), a natural language processing module (204), a phonetic emotion analysis module (205), and a demographic analysis module (203). Video data (208) is received by the facial expression recognition module (202) and the facial recognition module (244). Facial expression recognition data (213) is transmitted by the facial expression recognition module (202) and facial recognition data (245) is transmitted by the facial recognition module (244). Image data (209) is received by the demographic analysis module (203), which most commonly transmits age (505), race (506), and gender (507) that is depicted as separate streams but is often combined into a single data stream, demographic analysis data (215). Audio data (210) is received by an audio preprocessor (207). The audio preprocessor (207), shown being within the emotion and identity detection system (222), may not require a machine learning system to perform its functions, and will not be part of the emotion and identity detection system (222) in all embodiments. The audio preprocessor output (212) is directed to the natural language processing module (204) and the phonetic emotional analysis module (205). The natural language processing module (204) sends natural language output data (216) comprising but not limited to sentiment data (501), intent data (502), and entity recognition data (503). The phonetic emotional analysis module (205) transmits phonetic emotional analysis data (217).

In one embodiment, the facial expression recognition module (202), the demographic analysis module (203), and the facial recognition module (245) may each use a deep learning system to perform their functions, while the natural language processing module (204) and the phonetic emotional analysis module (205) may operate on a machine learning system.

Other embodiments may have all modules using a deep learning system or each using a machine learning system or combinations thereof. The facial recognition module (245) may have an embodiment that operates on a pattern recognition system rather than a machine learning system. The gaze tracking module (201) may run on a machine learning system but its most common embodiment does not require a machine learning system in order to perform its functions.

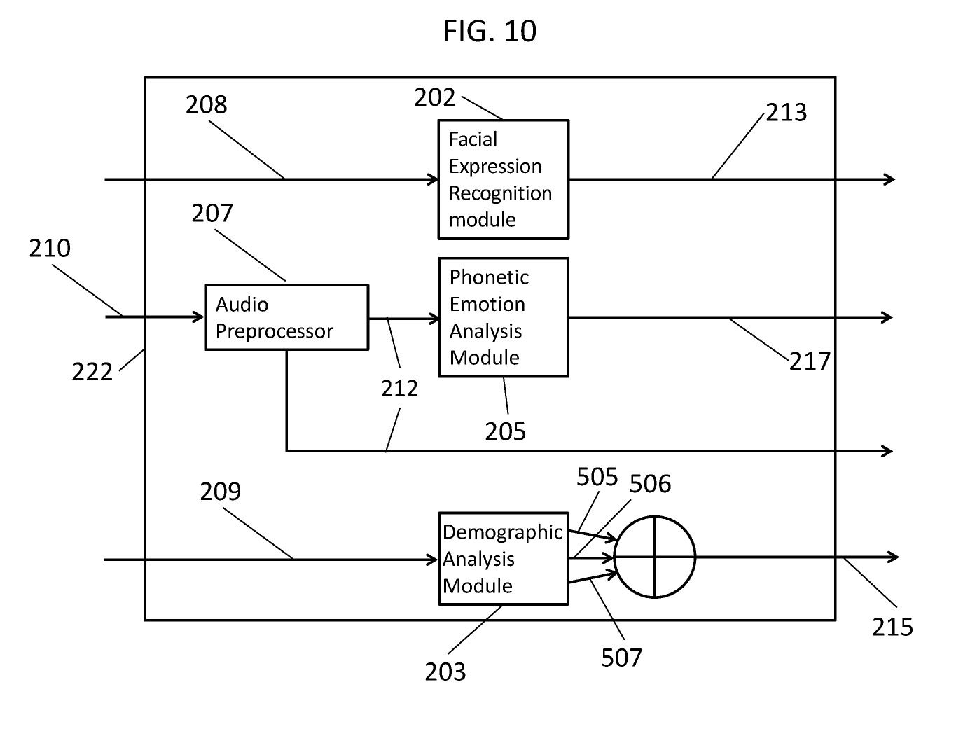

The embodiments in FIG. 9 and FIG. 10 may both be located on the data input device.

FIG. 10 depicts an emotion and identity detection system (222) embodiment that includes an audio preprocessor (207), a facial expression recognition module (202), a phonetic emotion analysis module (205), and a demographic analysis module (203). This embodiment may be located on the data input device (not shown), with natural language processing and facial recognition being done on a separate system. Natural language processing tends to be a more resource intensive process, and audio preprocessor data (212) can be transmitted to a natural language processing module located on a computing device that can devote more computing resources to performing the function. The facial recognition module is also not part of this embodiment because a machine learning system may not be necessary to perform facial recognition or it may be desirable to have an emotion and identity detection system (222) that uses less computing resources.

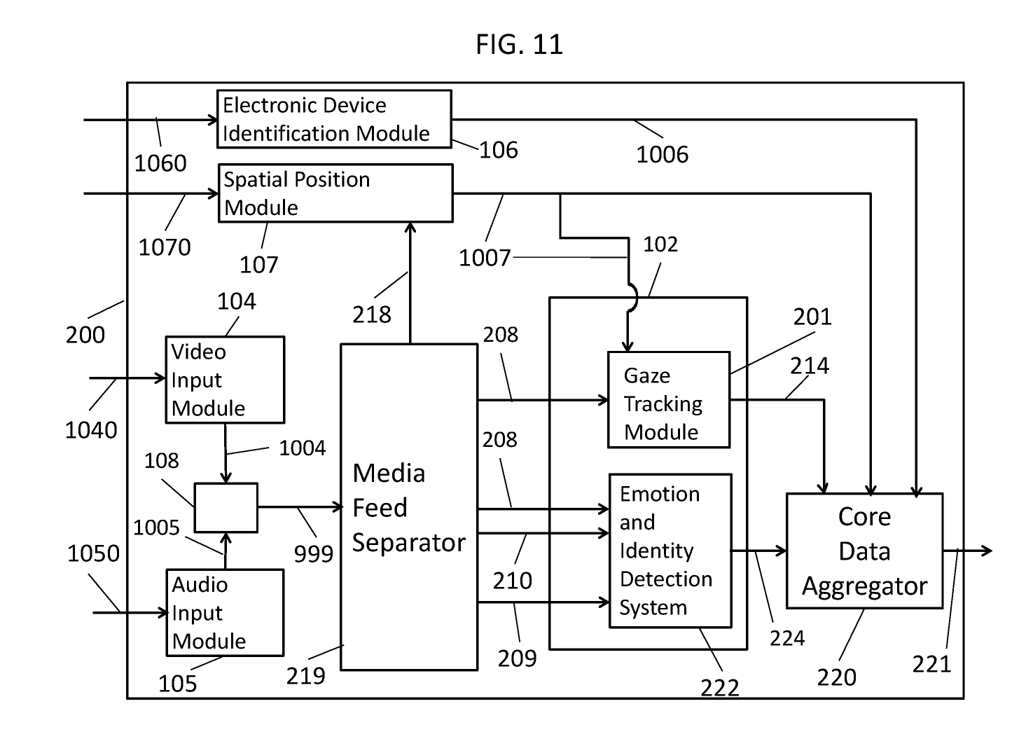

FIG. 11 shows an embodiment of data input device having components of a behavior learning system (102). This is also known as the core data input device (200). The embodiment has at least one gaze tracking module (201) and at least one emotion and identity detection system (222). At least some of the behavior learning analysis is performed within the data input device itself before sending the emotion and identity output data (221) to the network for further processing in the profile building system (not shown). The emotion and identity detection system (222) is commonly a computerized machine learning system that may have at least one facial expression recognition module, at least one facial recognition module, at least one demographic analysis module, at least phonetic emotional analysis module, at least one audio preprocessor module, at least one natural language processing module, and/or combinations. Further shown in this embodiment are a media feed separator (219) and a core data aggregator (220), which may be components of at least one data input device processor (not shown). Also shown are at least one video input module (104), at least one audio input module (105), at least one electronic device identification module (106) and at least one spatial position module (107), and at least one data input device processor (108).

In this embodiment of a core data input device (200), an electronic device signal input (1060) is received by the at least one electronic device identification module (106) and electronic device identification data (1006) is transmitted by the electronic device identification module (106) to the core data aggregator (220). Spatial position input (1070) is received by the at least one spatial position module (107) and spatial position data (1007) is transmitted by the spatial position module (107) to the gaze tracking module (102) and/or the core data aggregator (220). The at least one video input module (104) is shown receiving video input (1040) and providing video data (1004) as output to an input data processor (108). The at least one audio input module (105) is shown receiving audio input (1050) and providing audio data (1005) as output to the input data processor (108). The input data processor aggregates the audio and video streams, providing media (999). Media (999), comprising audio, video, and/or image data, is received by the media feed separator (219), where the data is separated and it is directed to the appropriate processor and/or module. In this case, video data (208), image data (209), and audio data (210) are directed to the emotion and identity detection system (222). Spatial video data (218) may be provided to the spatial position module (107). Video data (208) is also directed to the at least one gaze tracking module (201). Within the at least one gaze tracking module, video data (208) and spatial data (1007) are received and processed. Gaze tracking data (214) is directed by the at least one gaze tracking module (201) to the core data aggregator (220). The emotion and identity detection system (222) is a form of machine learning system. The combined output (224) of the modules (not shown) that comprise the emotion and identity detection system (222) is sent to the core data aggregator (220). The combined output (224) of the emotion and identity detection system (222) may comprise facial expression recognition data, facial recognition data, demographic analysis data, natural language output data, and/or phonetic emotional analysis data. The combined output (224) may be an individual or combined stream or both. The electronic device identification data (1006), the spatial position data (1007), the gaze tracking data (214), and the combined output (224), are processed by the core data aggregator (220) and emotion and identity output data (221) is sent to the profile building system (not shown). The emotion and identity output data (221) may comprise individual data streams, with each stream representing the electronic device identification data (1006), the spatial position data (1007), the facial expression recognition data (213), facial recognition data (245) the gaze tracking data (214), the demographic analysis data (215), the natural language output data (216), and/or the phonetic emotional analysis data (217). It may also be a combined stream or combinations of individual and combined streams.

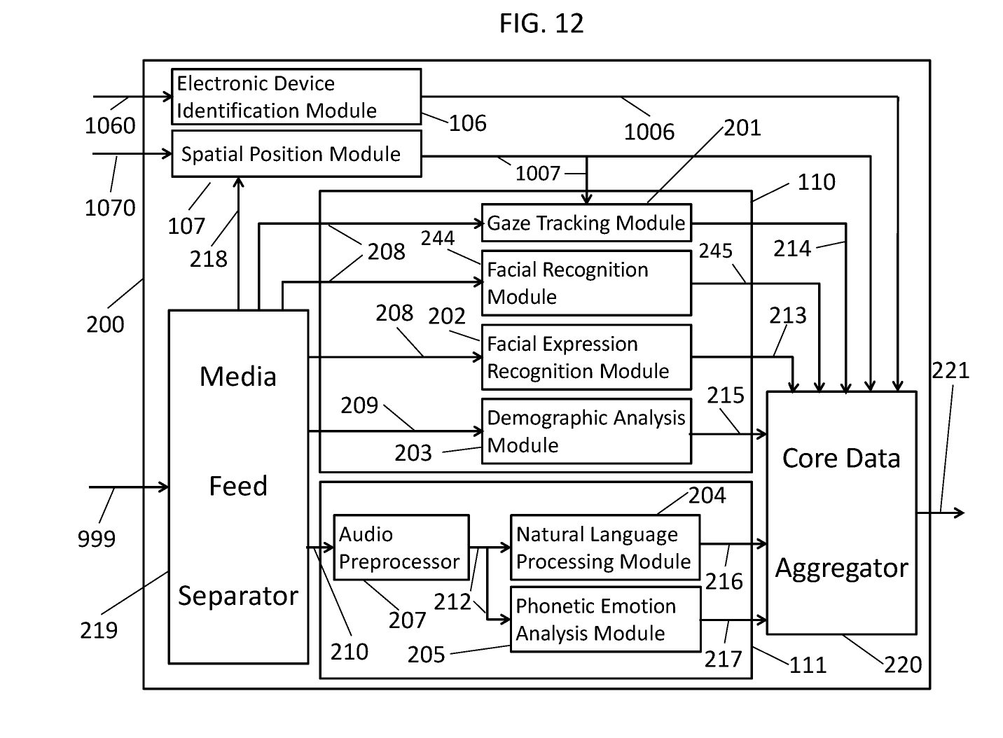

FIG. 12 depicts an embodiment of a data input device comprising components of a behavior learning system, or core data input device (200). This embodiment shows all components of the behavior learning system (102) within the data input device itself. This behavior learning system comprises at least one video data processor (110) and at least one audio data processor (111). The at least one video data processor (110) has at least one gaze tracking module (201), at least one facial recognition module (244), at least one facial expression recognition module (202), at least one demographic analysis module (203). The at least one audio data processor (111) has at least phonetic emotional analysis module (205), at least one audio preprocessor module (207), and at least one natural language processing module (204). Further shown in this embodiment are a media feed separator (219) and a core data aggregator (220), which may be components of at least one data input device processor. Also shown are at least one electronic device identification module (106) and at least one spatial position module (107).

In this embodiment of a core data input device (200), an electronic device signal input (1060) is received by the at least one electronic device identification module (106) and electronic device identification data (1006) is transmitted by the electronic device identification module (106) to the core data aggregator (220). Spatial position input (1070) is received by the at least one spatial position module (107) and spatial position data (1007) is transmitted by the spatial position module (107) to the gaze tracking module (201) and/or the core data aggregator (220). Media (999) comprising audio, video, and/or image data is received by the media feed separator (219), where the data is separated and it is directed to the appropriate processor and/or module. In this case, video data (208) and image data (209) are directed to components of the at least one video data processor (110). Spatial video data (218) may be provided to the spatial position module (107). Spatial video data (218) may include barcode information taken from an image or video of surrounding items or products, or from barcodes that are affixed near the products for the purpose of location determination. Such barcode information may be used to identify the absolute location of the data input device. Audio data (210) is directed to components of the at least one audio data processor (111). Within the video data processor (110), video data (208) is directed to the at least one gaze tracking module (201), at least one facial recognition module (244), and the at least one facial expression recognition module (202). Image data (209) is directed to the demographic analysis module (203). In this embodiment, image data (209) is derived from the video stream of the media (999). The image data (209) may be obtained from the media feed separator (219) or it may be obtained from a data input device processor (not shown), combined with the media (999), and separated and directed by the media feed separator (219). The at least one facial expression recognition module (202) sends facial expression recognition output data (213) to the core data aggregator (220). The at least one facial recognition module (244) sends facial recognition output data (245) to the core data aggregator (220). Within the at least one gaze tracking module, video data (208) and spatial position data (1007) is received and processed by the gaze tracking module (201). Gaze tracking data (214) is directed by the at least one gaze tracking module (201) to the core data aggregator (220). The demographic analysis module (203) processes image data (209) and provides demographic analysis data (215) to the core data aggregator (220). Within the audio data processor (111), audio data (210) is directed to the at least one audio preprocessor (207) where initial audio data (210) processing occurs. The audio preprocessor output (212) is directed to the natural language processing module (204) and the phonetic emotional analysis module (205). The natural language processing module (204) sends natural language output data (216) comprising but not limited to natural language understanding data, sentiment analysis data, and named entity recognition data, to the core data aggregator (220). The phonetic emotional analysis module (205) sends phonetic emotional analysis data (217) to the core data aggregator (220). The electronic device identification data (1006), the spatial position data (1007), the facial expression recognition data (213), the facial recognition data (245), the gaze tracking data (214), the demographic analysis data (215), the natural language output data (216), and the phonetic emotional analysis data (217), are processed by the core data aggregator (220) and emotion and identity output data (221) is sent to the profile building system (not shown). The emotion and identity output data (221) may have individual data streams, with each stream representing the electronic device identification data (1006), the spatial position data (1007), the facial expression recognition data (213), the facial recognition data (245), the gaze tracking data (214), the demographic analysis data (215), the natural language output data (216), and the phonetic emotional analysis data (217) or it may be a combined stream or combinations of individual an combined streams.

A more general embodiment of the core data input device (200) depicted may have at least one, some, or all of the modules that make up the video data processor (110) and the audio data processor (110) and thus the behavior learning system. This is an embodiment where the behavior learning system is within the data input device.

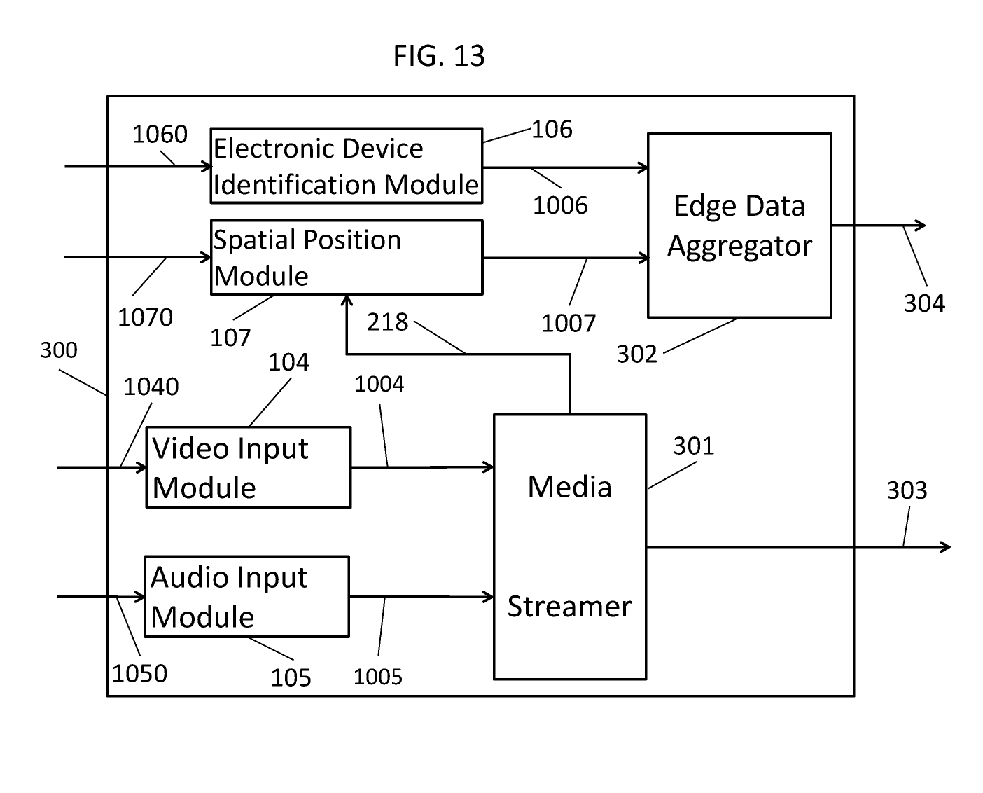

FIG. 13 depicts an embodiment of a data input device known as the edge data input device (300). Shown are at least one video input module (104), at least one audio input module (105), at least one electronic device identification module (106), and at least one spatial position module (107). The at least one video input module (104) is shown receiving video input (1040) and providing video data (1004) as output. The at least one audio input module (105) is shown receiving audio input (1050) and providing audio data (1005) as output. The at least one electronic device identification module (106) is shown receiving electronic device signal input (1060) and providing electronic device identification data (1006) as output. The at least one spatial position module (107) is shown receiving spatial position input (1070) and providing spatial position data (1007). Also shown are an edge data aggregator (302) and a media streamer (301). The edge data aggregator (302) processes electronic device identification data (1006) and spatial position data (1007) and combines the data into a single stream, aggregated spatial and electronic device identification data (304). The media streamer (301) receives video data (1004) and audio data (1005) and will stream the streamed media data (303). The streamed media data (303) is depicted by a single output arrow but the streamed media data (303) may be aggregated or be separate data streams. The edge data aggregator (302) and the media streamer (301) may be a single data input device processor or multiple processors.

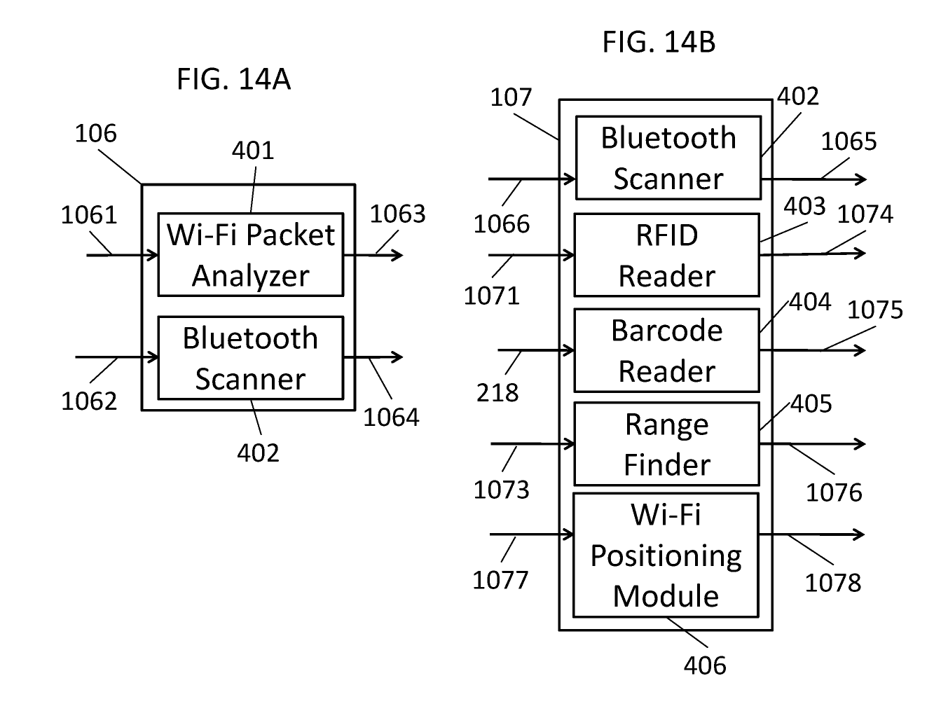

FIG. 14A depicts an embodiment of an electronic device identification module (106). The electronic device identification module (106) may comprise a Wi-Fi packet analyzer (401) and/or a Bluetooth® scanner (402). Wi-Fi input (1061) is received by the Wi-Fi packet analyzer (401) and Wi-Fi identification data (1063), most commonly in the form of a MAC ID, is transmitted. Bluetooth® input (1062) is received by the Bluetooth® scanner (402) and Bluetooth® mobile electronic device address data (1064) is transmitted. Bluetooth® input (1062) includes Bluetooth® mobile electronic device address data (1064), and is used to uniquely identify a mobile electronic device.

FIG. 14B depicts an embodiment of a spatial position module (107). The spatial position module (107) may comprise an RFID reader (403) and/or a barcode reader (404) and/or a range finder (405) and/or a Bluetooth® scanner (402), and/or a Wi-Fi positioning module (406). The RFID reader (403) receives RFID signal data (1071) and transmits RFID output (1074), most commonly in the form of an RFID tag number that encodes product location information, which is used to determine data input device location. The barcode reader (404) may receive video or image data input (218) and will transmit barcode data (1075), most commonly in the form of barcode encoded product location information, which is used to determine data input device location. The Bluetooth® scanner (402) receives Bluetooth® Low Energy (BLE) beacon input (1066) and BLE data (1065) is transmitted. Bluetooth® Low Energy (BLE) beacon input (1066) may come from a plurality of surrounding beacons, in the form of beacon identification and/or encoded location information. The closest beacon is determined by the Bluetooth® scanner (402) and BLE data (1065) is transmitted, with the BLE data (1065) having beacon identification information and/or encoded location information. The range finder (405) receives range input (1073) from a passing person and transmits range data (1076), in the form of height, horizontal distance, and other range data as needed, determining absolute position data, relative position data, height data, and horizontal distance data. Most commonly, the range finder gathers range input (1073) using laser sensors, and/or ultrasonic sensors, and/or infrared sensors; however other electromagnetic radiation gathering sensors may be used. The spatial position module (107) may serve to gather the absolute location of the data input device, and/or data input device location relative to the location in which the data input devices are placed, and/or data input device location relative to the surrounding items, and/or spatial measurements related to the person within range of the range finder (405).

Wi-Fi positioning is another option for determining the location of the data input device. Common methods for Wi-Fi positioning include: received signal strength indication, fingerprinting, angle of arrival, and time of flight based techniques for location determination. The data input device is linked to a network and based on that network link, the device position may be determined. If Wi-Fi positioning is being used, then the Wi-Fi positioning module (406) may receive network Wi-Fi signal data (1077) and may transmit Wi-Fi positioning data (1078), most commonly in the form of data input device location.

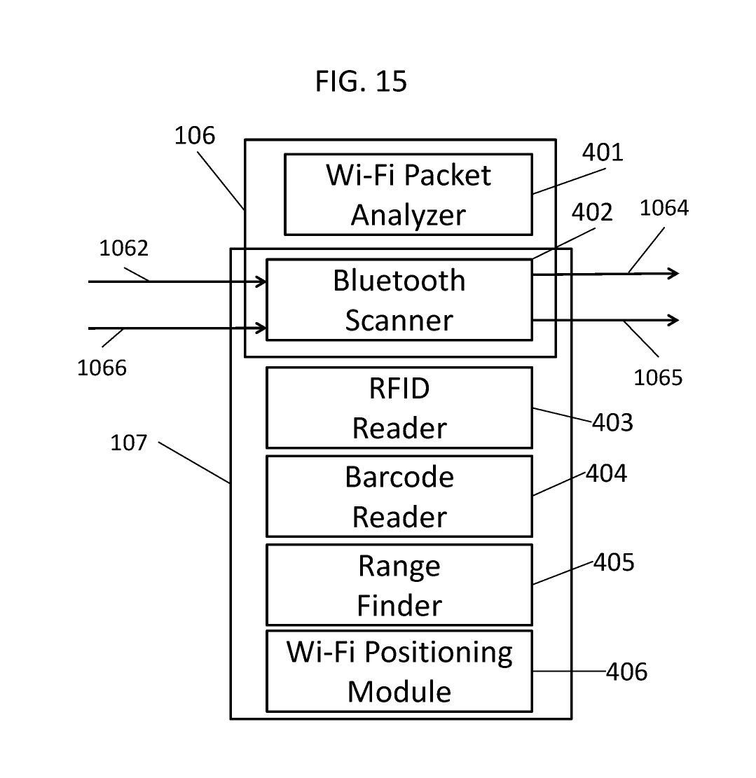

FIG. 15 depicts a single Bluetooth® scanner (402) shared by the electronic device identification module (106) and the spatial position module (107). In a data input device where Bluetooth® data is collected by both the electronic device identification module (106) and the spatial position module (107) the Bluetooth® scanner (402) may be a single scanner that performs a dual function, meeting the requirements for both the electronic device identification module (106) and the spatial position module (107). Bluetooth® devices may gather and transmit both standard Bluetooth® and BLE signals. In this embodiment, the Bluetooth® scanner (402) receives Bluetooth® input (1062) and Bluetooth® mobile electronic device address data (1064) is transmitted. The Bluetooth® scanner (402) also receives BLE beacon input (1066) and BLE data (1065). BLE data is transmitted and used to identify the location of the data input device.

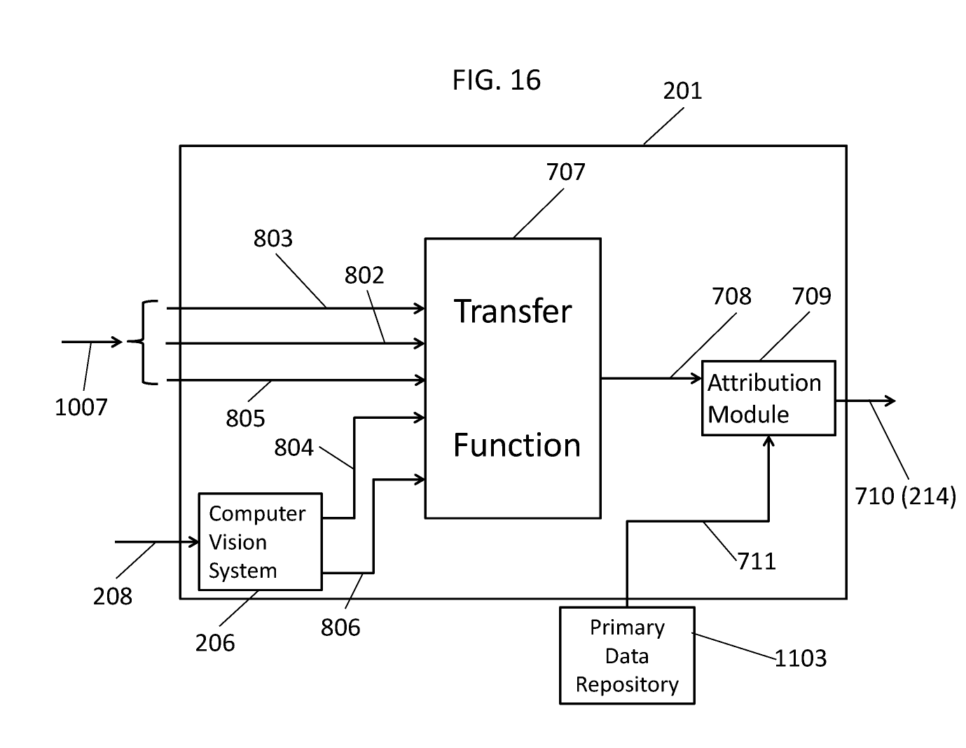

FIG. 16 depicts a gaze tracking module (201). In this embodiment the gaze tracking module comprises a computer vision system (206), a transfer function module (707), and an attribution module (709). The computer vision system (206) receives and processes video data (208), and transmits eye position (804) and head orientation (806) to the transfer function module (707). The eye position (804) refers to data that includes the Cartesian coordinates (x, y) of the subject's eyes on a vertical plane. The head orientation (806) refers to the yaw, pitch and roll angles of a subject's head in a three dimensional space along the normal, lateral and longitudinal axes. In this embodiment, spatial position data (1007) includes horizontal distance data (802), video input device field-of-view data (803), and height above the floor data (805). Field-of-view data (803) is the field of view of a data input device (not shown). The horizontal distance data (802) includes the distance to a subject within the field of view of the data input device. The height above the floor data (805) is the height of a data input device above a solid flat horizontal surface. The horizontal distance data (802), video input device field-of-view data (803), height above the floor data (805), eye position (804) and head orientation (806) is received by the transfer function module (707). The transfer function module (707) processes input data, performing mathematical calculations on the input data to determine a user's field of view, and transmits user field of view data (708) to the attribution module (709). The attribution module (709) retrieves planogram data (711) and receives the user field of view data (708). Human field of view data, while similar to the data input device's field-of-view, is calculated to determine the gaze direction of the subject, rather than the field of view of the data input device directed towards the subject. The attribution module (709) processes data, to determine the items the user is looking at, and transmits gaze tracking data (214), which in a retail location may be in the form of target merchandise data (710). The tracking data (214) is a gaze tracking vector which indicates where a subject is looking and can be used to determine what a subject is looking at. In a retail environment, the gaze tracking vector is used to identify merchandise viewed by a subject. Planogram data (711), containing product location information, may be retrieved from at least one primary data repository (1103). Gaze tracking is commonly performed through a computer calculation based on video input and spatial position input. There are embodiments that may use a machine learning system to determine a subject's field-of-view and to identify items viewed by the subject, in the role of the computer vision system (206).

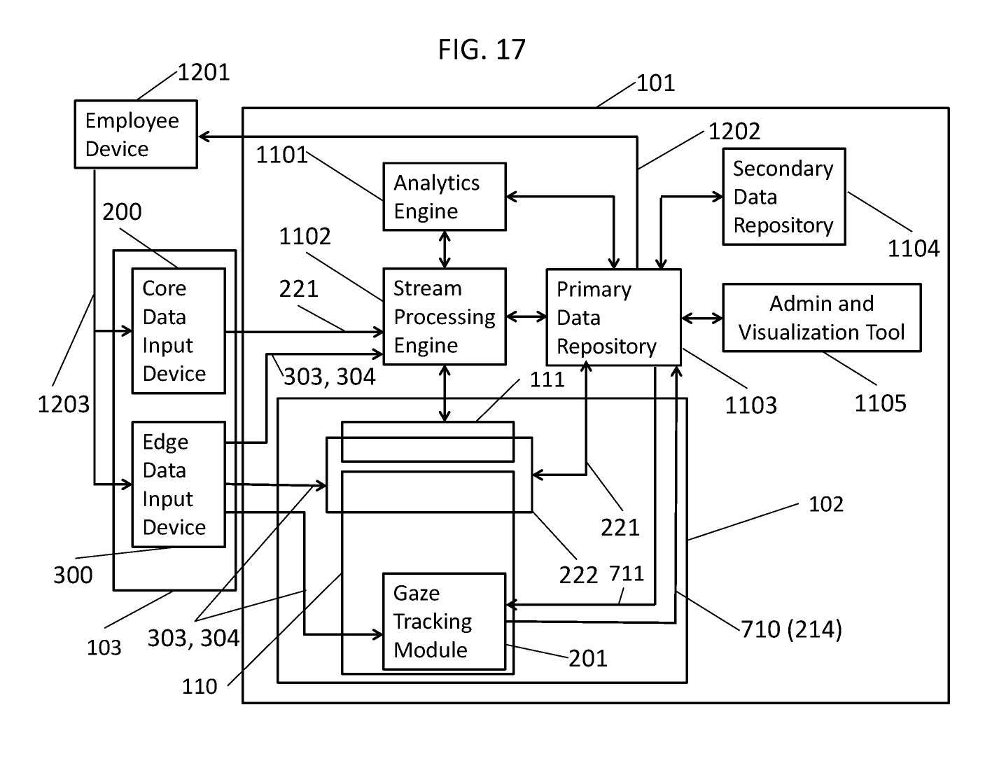

FIG. 17 depicts an embodiment of a distributed system for building a plurality of user profiles and network. The distributed system has at least one data input devices which may be at least one edge data input device (300) and/or at least one core data input device (200). Both an edge data input device (300) and a core data input device (200) are shown. A distributed system for building a plurality of user profiles may have multiple core data input devices (200), with embodiments of the core data input device (200) having at least one, some, or all of the modules that make up the behavior learning system (102). A distributed system for building a plurality of user profiles may have multiple edge data input devices (300). In this embodiment, at least one data input device (103) is represented by the core data input device (200), comprising all behavior learning system modules, and the edge data input device (300). The at least one data input device (103) transmits data to a profile building system (101). The profile building system (101) comprises a behavior learning system, with at least one machine learning modules depicted by the emotion and identity detection system (222). A video data processor (110) and an audio data processor (111) are shown intersecting with the emotion and identity detection system (222). The emotion and identity detection system (222) comprises at least one machine learning system, which is commonly required by some of the behavior learning system modules. The video data processor (110) also may have a gaze tracking module (201). The profile building system (101) further has at least one stream processing engine (1102), at least one analytics engine (1101), at least one primary data repository (1103), at least one secondary data repository (1104), and at least one administration and visualization tool (1105).

Video and audio data is transmitted from the core data input device (200) transmitting emotion and identity output data (221) to at least one stream processing engine (1102). The emotion and identity output data (221) comprises output from all behavior learning system (102) modules. No further direct processing is required by the behavior learning system (102) in the profile building system (101). Further shown, the at least one edge data input device (300) transmits streamed media data (303) and aggregated spatial and electronic device identification data (304) to the emotion and identity detection system (222), the gaze tracking module (201), and the at least one stream processing engine (1102). Streamed media data (303) and aggregated spatial and electronic device identification data (304) are shown as a single stream.

The at least one stream processing engine (1102) analyzes and processes data in real-time, continuously calculating mathematical or statistical analytics, using input from the analytics engine (1101), and transmitting stream processing output data to an appropriate engine and/or system for further processing and/or analysis and/or storage. The at least one stream processing engine (1102) is shown communicating with an emotion and identity detection system, at least one primary data repository (1103), and at least one analytics engine (1101). The at least one analytics engine (1101) provides descriptive, predictive, and prescriptive analytics and identifies qualitative or quantitative data patterns, communicating this information to the stream processing engine (1102). The at least one analytics engine (1101) communicates with the at least one stream processing engine (1102) and the at least one primary data repository (1103). The at least one primary data repository (1103) communicates with the emotion and identity detection system (222), the gaze tracking module (201), the stream processing engine (1102), the analytics engine (1101), the at least one secondary data repository (1104), and the at least one administration and visualization tool (1105). The at least one primary data repository may receive emotion and identity output data (221) directly from the emotion and identity detection system (222) and gaze tracking data or target merchandise (710, 214) from the at least one gaze tracking module (201). The gaze tracking module (201) may receive planogram data. The administration and visualization tool (1105) provides reporting and system management tools.

Since a subject moves through or about a fixed space, the subject may move from one device to another, or from an area with core data input devices (200) to an area of the fixed space with edge data input devices (300). The stream processing engine (1102) will help to coordinate updates to the primary data repository (1103) of a moving subject passing from one data input device to the next and passing between data input devices that may gather different types of input data.

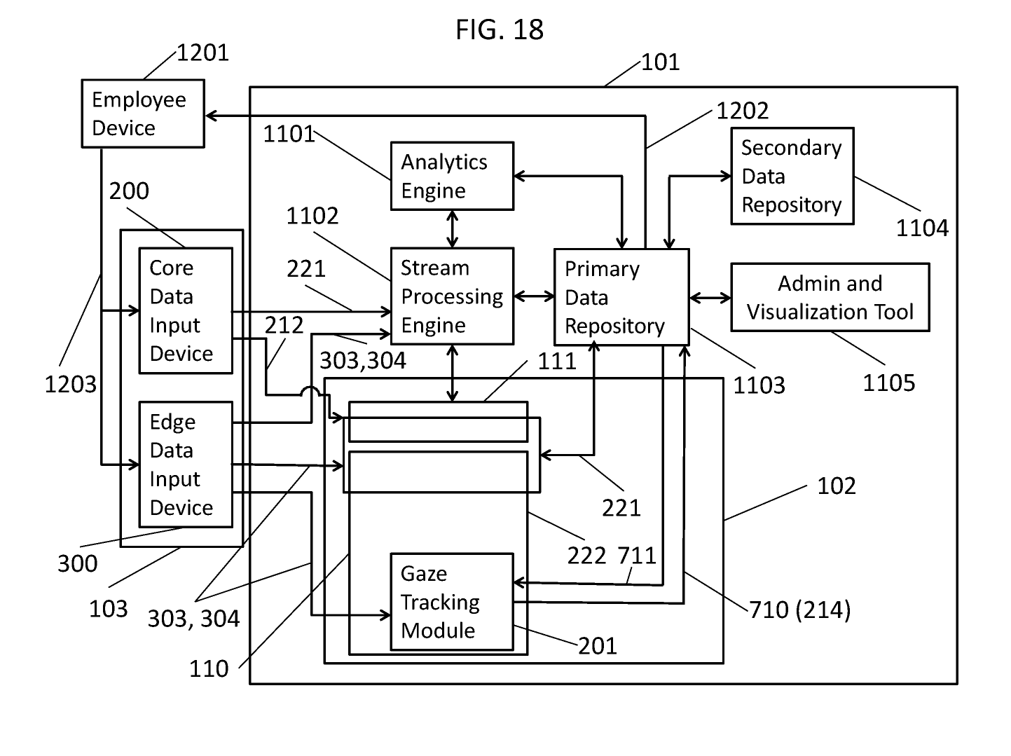

FIG. 18 depicts the distributed system for building a plurality of user profiles and network of FIG. 17, with the core data input device (200) having a behavior learning system (102) without a natural language processing module (204). The core data input device (200) directs audio preprocessor output (212) to a natural language processing module (204) located in the audio processor (111) within the behavior learning system (102) within the profile building system (101).

The emotion and identity output data (221) comprises output from behavior learning system (102) modules. The stream processing engine (1102) communicates with the behavior learning system (102) on the profile building system (101) and may coordinate updates and transmissions to the primary data repository (1103).

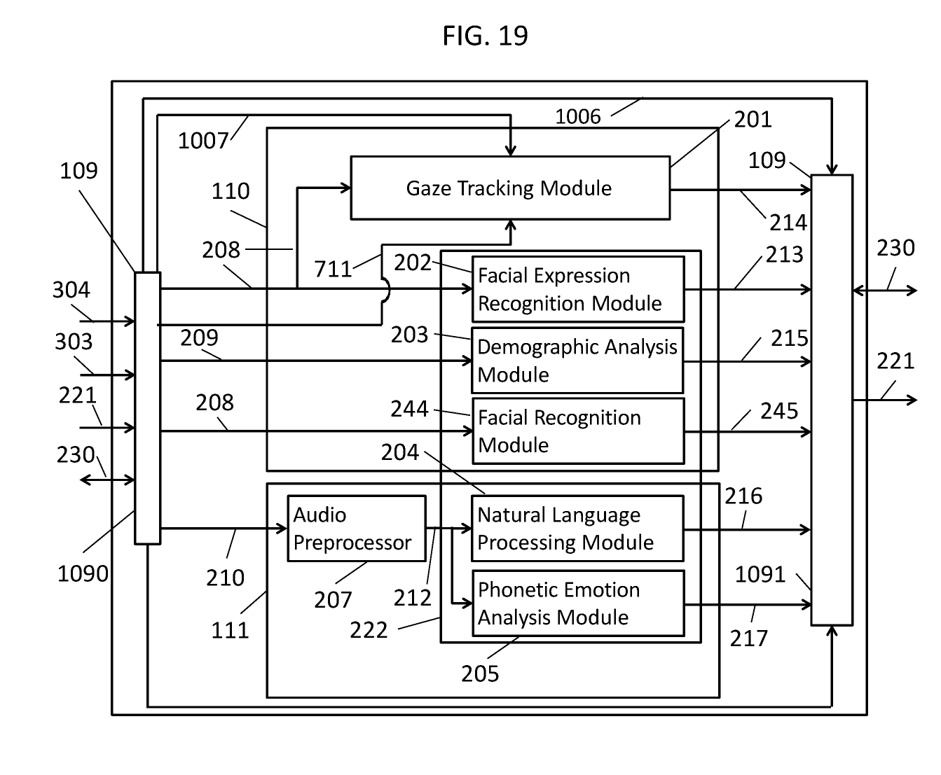

FIG. 19 depicts an embodiment of a behavior learning system (102). This behavior learning system (102) comprises at least one behavior learning processor (109), at least one video data processor (110) and at least one audio data processor (111). The at least one video data processor (110) has at least one gaze tracking module (201), at least one facial expression recognition module (202), at least one demographic analysis module (203). The at least one behavior learning processor (109) may include but is not limited to devices that provide data aggregation, data streaming, data separation, and combinations thereof. Shown are a first behavior learning processor (1090) and a second behavior learning processor (1091). The at least one audio data processor (111) has at least phonetic emotional analysis module (205), at least one audio preprocessor module (207), and at least one natural language processing module (204). Further shown is at least one emotion and identity detection system (222). The at least one facial expression recognition module (202), the at least one demographic analysis module (203), the at least one phonetic emotional analysis module (205), and the at least one natural language processing module (204) are all components of the at least one emotion and identity detection system (222). The audio preprocessor (207) may be within or outside the emotion and identity detection system (222) but it is shown outside in this figure.

In this embodiment streamed media data (303), aggregated spatial and electronic device identification data (304), emotion and identity output data (221), and stream processing engine data (230), comprising audio, video, spatial, electronic device identification data, and/or image data are received by the first behavior learning processor (1090), where the data processed and it is directed to the appropriate processor and/or module. Stream processing engine data (230) is data exchanged between the behavior learning system (102) and the stream processing engine (not shown). Electronic device identification data (1006) is directed by the first behavior learning processor (1090) for further processing. Video data (208), spatial position data (1007), planogram data (711), and image data (209) are directed to components of the at least one video data processor (110), and the audio data (210) is directed to components of the at least one audio data processor (111). Within the video data processor (110), video data (208), planogram (711), and spatial position data (1007) is directed to the at least one gaze tracking module (201) and video data (208) the at least one facial expression recognition module (202), and image data (209) is directed to the demographic analysis module (203). The at least one facial expression recognition module (202) sends facial expression recognition output data (213) to the second behavior learning processor (1091) for further processing and directing. The at least one gaze tracking module receives video data (208), spatial position data (1007), and/or planogram data (711). Gaze tracking data (214) is directed by the at least one gaze tracking module (201) to the second behavior learning processor (1091) for further processing and directing. Within the audio data processor (111), audio data (210) is directed to the at least one audio preprocessor (207) where initial audio data (210) processing occurs. The demographic analysis module (203) processes image data (209) and provides demographic analysis data (215) to the second behavior learning processor (1091) for further processing and directing. The audio preprocessor output (212) is directed to the natural language processing module (204) and the phonetic emotional analysis module (205). The natural language processing module (204) sends natural language output data (216) comprising but not limited to natural language understanding data, sentiment analysis data, and named entity recognition data, to the second behavior learning processor (1091) for further processing, and directing. The phonetic emotional analysis module (205) sends phonetic emotional analysis data (217) to the second behavior learning processor (1091) for further processing, and directing. The electronic device identification data (1006), the spatial position data (1007), the facial expression recognition data (213), the gaze tracking data (214), the demographic analysis data (215), the natural language output data (216), and the phonetic emotional analysis data (217), are processed by the second behavior learning processor (1091) and emotion and identity output data (221) is sent to the at least one primary data repository (not shown) and/or stream processing engine data (230) is communicated to the stream processing engine (not shown). The emotion and identity output data (221) may have individual data streams, with each stream representing the electronic device identification data (1006), the spatial position data (1007), the facial expression recognition data (213), the gaze tracking data (214), the demographic analysis data (215), the natural language output data (216), and the phonetic emotional analysis data (217), or it may be a combined stream, or combinations of individual an combined streams.

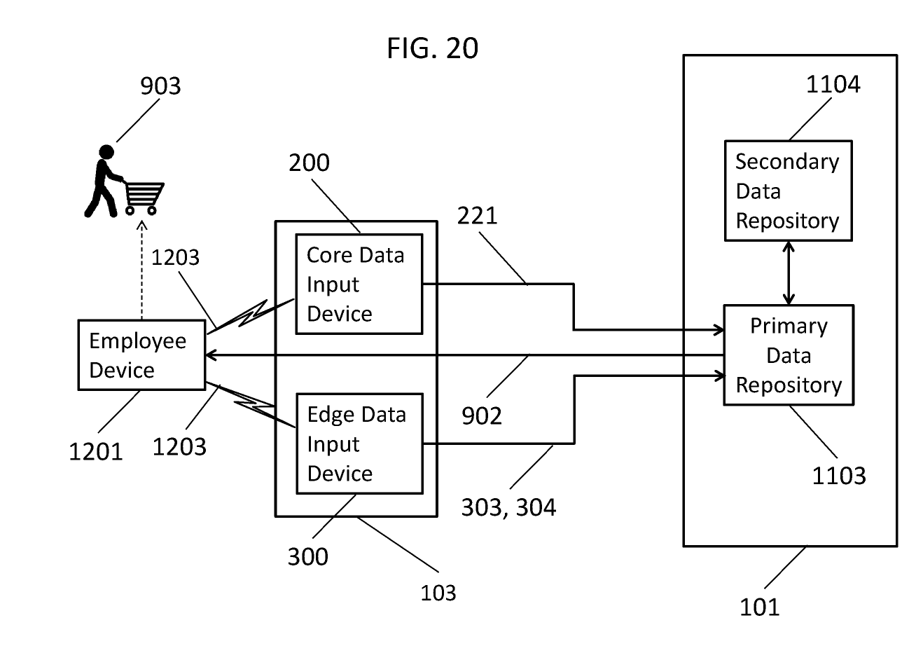

FIG. 20 depicts an embodiment of the communication stream for at least one employee interface device (1201) for a retail setting. Shown are at least one shopper (903), at least one data input device (103) represented by a core data input device (200) and an edge data input device (300). Also shown is a profile building system (101) with at least one primary data repository (1103) and at least one secondary data repository (1104). The employee interface device (1201) communicates data input device instructions (1203) with the at least one data input device (103). The employee interface device (1201) communication includes but is not limited to instructions, setup or provisioning, feedback, alarms, status, location, and maintenance. The at least one data input device (103) transmits combined emotion and identity output (221) and/or streamed media data (303) and aggregated spatial and electronic device identification data (304) to the at least one primary data repository (1103). At least one secondary data repository (1104), storing secondary data, communicates with the at least one primary data repository (1103) and primary and secondary information may be combined as required. The profile building system (101) transmits employee interface device instruction data (902) from the primary data repository (1103) to the employee interface device (1201), where it is processed and displayed to the employee. The employee may be instructed to approach the shopper (903) with suggestions or special offers for products. The employee may also be provided with security instructions or security personnel may be alerted. The user profile helps the retailer generate a customer profile, which allows the retailer to provide the customer with an enhanced or even customized experience. In exchange, the retailer is able to collect data on physical visitors which may ordinarily only be available in an online shopping environment or through targeted market research, such as focus groups.

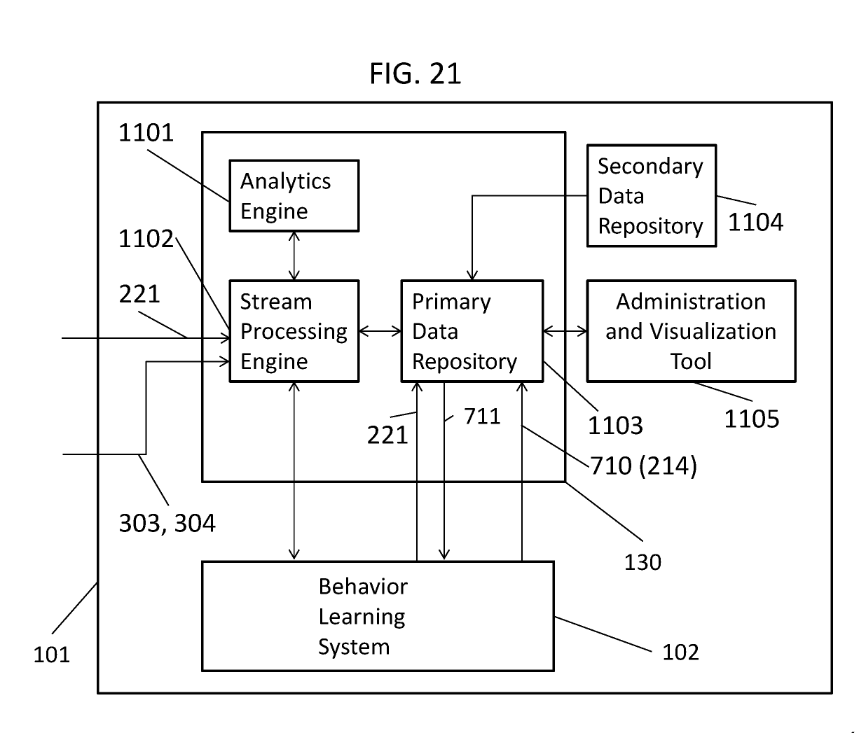

FIG. 21 depicts an embodiment of a profile building system (101). Shown are a behavior learning system (102), a behavioral response analysis system (130), at least one secondary data repository (1104), and an administration and visualization tool (1105). The behavior response analysis system (130) has at least one stream processing engine (1102), at least one analytics engine (1101), and at least one primary data repository (1103). Emotion and identity output data (221), streamed media data (303), and aggregated spatial and electronic device identification data (304) are show directly being received by the stream processing engine (1102). The stream processing engine (1102) is also shown communicating with the data analytics engine (1101), the behavior learning system (102), and the primary data repository (1103). The behavior learning system (102) is shown transmitting emotion and identity output data (221) and gaze tracking data (214), where the gaze tracking data (214) may be in the form of target merchandise data (710). The primary data repository (1103) is shown transmitting planogram data (711) to the behavior learning system (102) and receiving input from the secondary data repository (1104). While this embodiment refers to planogram data in general, the primary data repository (1103) may store planogram data (711) from multiple fixed-space locations but will retrieve planogram data (711) specific to the fixed-space in which the data input device is located. The primary data repository (1103) is also shown communicating with the stream processing engine (1102) and the administration and visualization tool (1105).

The at least one primary data repository (1103) may be a distributed database, a computational cluster, or an electronic mass data storage system for storing, organizing, and analyzing large amounts of structured or unstructured data, or combinations of mass data storage systems. For this system, common data options include but are not limited to, a Hadoop Cluster, a relational database management system, or a NoSQL framework of database. The at least one secondary data repository (1104) is a repository for market research or subject data which was obtained from a source outside the distributed system for building a plurality of user profiles (100), but the data may be available for use. The secondary data repository (1104) may be any type of mass storage system connected to and communicating with the distributed system for building user profiles. The at least one primary data repository (1103) and the at least one secondary data repository (1104) may physically be located within the same electronic mass data storage system or they may be located on different electronic mass data storage systems. A plurality of user profiles are to be stored within the at least one primary data repository (1103). A user profile from the plurality of user profiles may comprise an assortment of data, to be determined by each individual retailer. However, the user profile may contain data selected from the emotion and identity output data (221) and/or the facial expression recognition data (213) and/or the gaze tracking data (214) and/or the demographic analysis data (215) and/or the natural language output data (216) and/or the phonetic emotional analysis data (217) and/or facial recognition data, and/or product purchase confirmation.

The behavior learning system (102) may put data directly into the at least one primary data repository (1103) or it may communicate with the behavior response analysis system (130) before directly writing data into the primary data repository (1103) or before sending data to the behavior response analysis system (130). The stream processing engine (1102) acts on a continual stream of data from at least one data input device, at least one behavior learning system, or from at least one data repository. It also communicates with at least one analytics engine to receive input on data handling.

As its primary purpose, the at least one analytics engine provides a business platform covering descriptive, predictive and prescriptive analytics solutions; it identifies qualitative or quantitative patterns in the users' structured or unstructured data through machine learning algorithms for facial recognition, facial expression recognition, age/race/gender determination, natural language processing, and phonetic emotion analysis; and it reports the analytics results.

An administration and visualization tool (1105) may provide reporting information to store managers or system administrators in textual and/or visual format. This data may be reported in an automatic fashion and/or also upon demand through queries with a specific set of criteria or parameters. System administrators can make manual adjustments to the system. In a retails setting, reporting data can be customized to the retailer or retailer location but will generally include demographic analysis data, and/or emotional analysis data, and/or intent data, and/or traffic data, and/or visit frequency data, and/or spending data, and/or heat map, and/or queue analysis data, and/or traffic analysis data, and/or people count data. Management tools may include but are not limited to an identity and access management tool, and/or an address resolution protocol table export tool, and/or a visitor characteristics tool, and/or a merchandise tool, and/or a planogram tool.

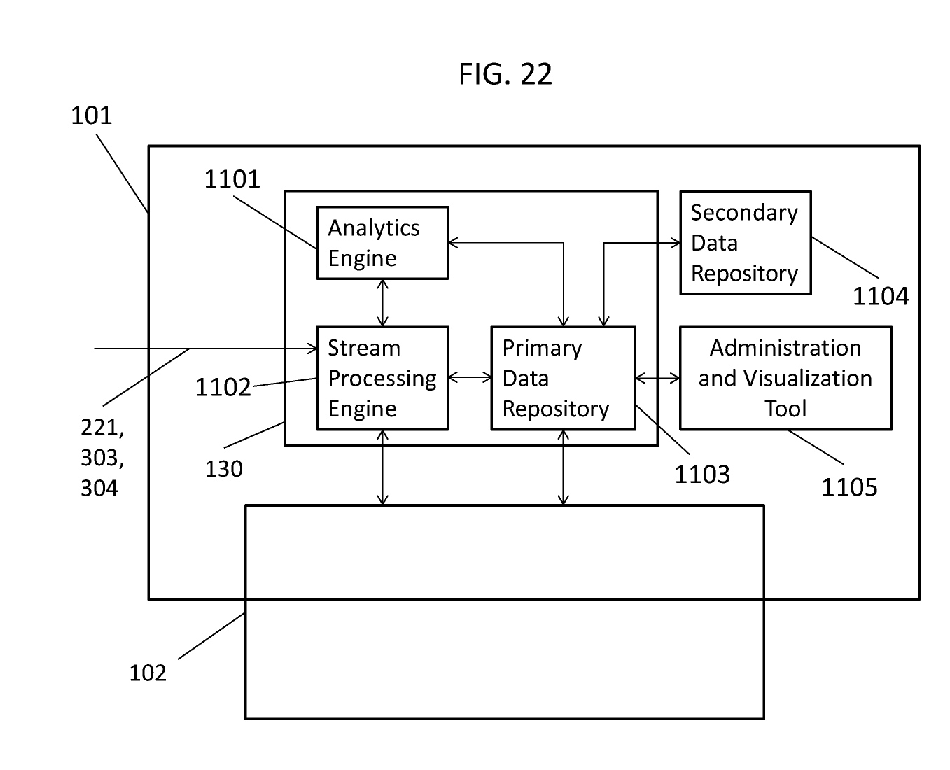

FIG. 22 depicts an embodiment of the profile building system (101), similar to FIG. 21. Part of a behavior learning system (102) block is depicted within the profile building system (101) and part of the behavior learning system (102) is located outside. There may be multiple behavior learning systems (102) updating a single primary data repository (1103) or the behavior learning system (102) may be physically located on a machine or machines apart from the profile building system (101). Also shown are a behavioral response analysis system (130), at least one secondary data repository (1104), and an administration and visualization tool (1105).

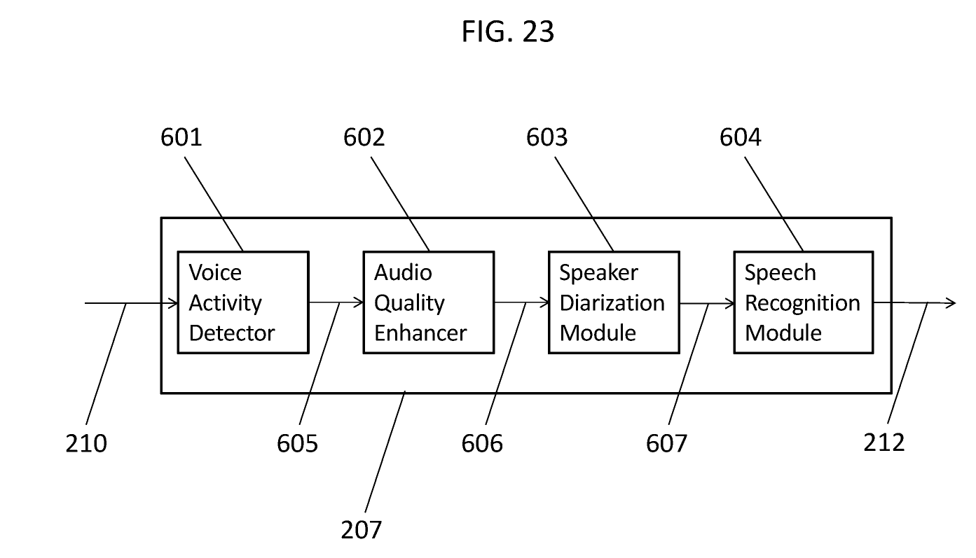

FIG. 23 depicts an embodiment of an audio preprocessor (207). Audio output from a data input processor and/or a behavior learning processor (109) is received by the audio preprocessor (207), for further processing. An audio processor comprises a voice activity detector (601), and/or an audio quality enhancer (602), and/or a speaker diarization module (603), and/or a speech recognition module (604). A common processing sequence includes but is not limited to processing by a voice activity detector (601), transmitting voice activity detector output (605) to an audio quality enhancer (602), transmitting enhanced audio quality data (606) to a speaker diarization module (603), transmitting speaker diarization output (607) to a speech recognition module (604), which transmits audio preprocessor output (212).

If a natural language processing module (204) is on the data input device (103), as depicted in FIG. 12, then all audio preprocessor steps are likely to be required and will comprise the audio preprocessor output (212).

If a phonetic emotional analysis module (205) is on the data input device (103) and natural language processing is performed on the profile building system (101) or within a separate behavior learning system (102), then the audio preprocessor (207) located on the data input device (103) may only require processing by a voice activity detector (601), transmitting voice activity detector output (605) to an audio quality enhancer (602), transmitting enhanced audio quality data (606) to a speaker diarization module (603), transmitting speaker diarization output (607), where the diarization output is the audio preprocessor output (212). A second audio preprocessor (not shown) located with the natural language processing module (204) may be required to receive audio preprocessor output (212) in the form of diarization output (607), and to perform speech recognition in the speech recognition module (604).

A voice activity detector captures and processes audio between periods of silence.

An audio quality enhancer provides additional signal processing operations such as beamforming, dereverberation, and ambient noise reduction to enhance the quality of the audio signal.

Diarization is the process of partitioning an input audio stream into homogeneous segments according to subject speaker identity. This method is used to isolate and categorize multiple audio streams coming from different subjects in a group conversation.

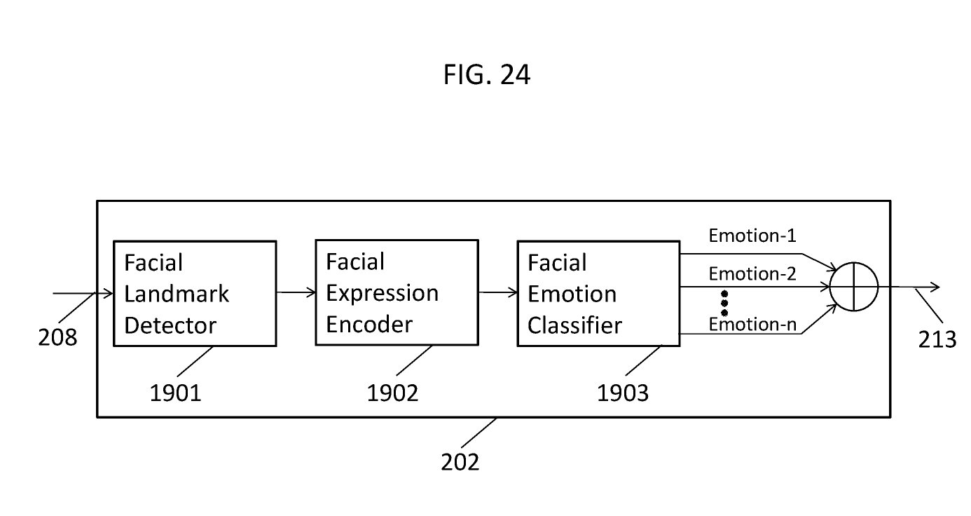

FIG. 24 depicts an embodiment of a facial expression recognition module (202) showing a facial landmark detector (1901), a facial expression encoder (1902), and a facial emotion classifier (1903). Video output (208) is transmitted to and received by the facial landmark detector (1902) for processing. The output from the facial landmark detector (1901) is transmitted to and received by the facial expression encoder (1902), where it is processed further. The output from the facial expression encoder (1902) is transmitted to and received by the facial emotion classifier, where it is processed. The output from the facial expression classifier (1903) is the facial expression recognition output data (213), which includes: a single data stream with at least one emotion but commonly multiple emotions, feedback on the subject's experience, and a scaled determination of emotional intensity.

Facial expression recognition is a method for gauging a subject's expression, including but not limited to, detecting and classifying emotions, detecting subject experience feedback, and providing engagement metrics to determine emotional intensity. A common embodiment has seven emotional classes, including: joy, anger, surprise, fear, contempt, sadness, disgust. A subject's experience feedback may involve calculating an emotional metric and determining the result on a scale between positive and negative endpoints. Engagement metrics are often used to determine emotional intensity on a scale between no expression and fully engaged endpoints.

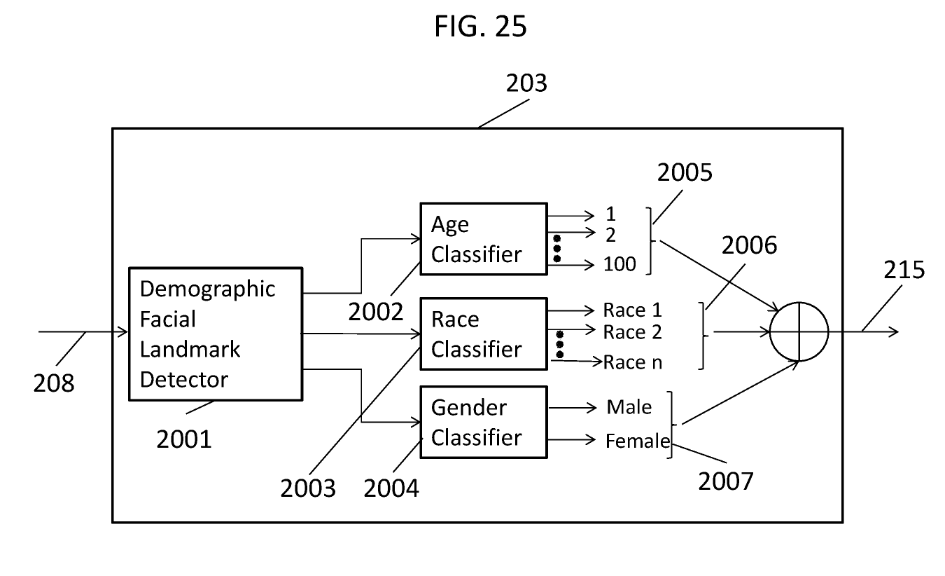

FIG. 25 depicts a demographic analysis module (203). Shown are a demographic facial landmark detector (2001), an age classifier (2002), a race classifier (2003), and a gender classifier (2004). Video output (208) is transmitted to and received by the demographic facial landmark detector (2001), where landmark data for a facial image is determined, and the output is transmitted to and received by an age classifier (2002), a race classifier (2003), and a gender classifier (2004). The age classifier determines a person's age, and provides age output (2005). Age can be either a specific number, or an estimated range. The race classifier (2003) determines a person's race and provides race output (2006). The gender classifier (2004) determines a person's gender and provides gender output (2007). Age output (2005), race output (2006), and gender output (2007) are generally transmitted as a single output stream, demographic analysis data (215).

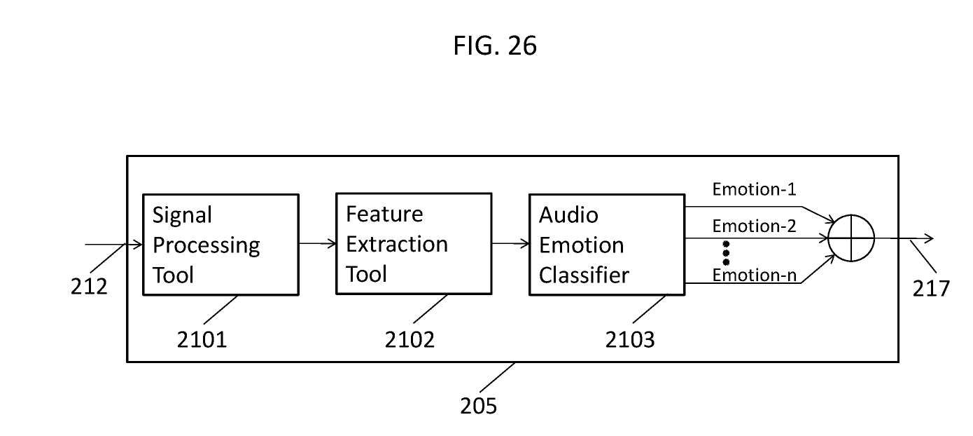

FIG. 26 depicts a phonetic emotional analysis module (205). Phonetic emotional analysis is a method of determining speech emotion and classifying that emotion. Audio preprocessor output data is received by the phonetic emotional analysis module (205) where a signal processing tool (2101) processes audio data and transmits signal process output data to a feature extraction tool (2102). The feature extraction tool (2102) further processes audio data and transmits phonetic feature and linguistic attribute data to an audio emotion classifier (2103). Phonetic features may include, volume, tone, tempo, pitch, intensity, prosody, simultaneous crosstalk between people, inflection, laughter, and sighs. Linguistic attributes include, words, pauses, silence, hesitation, inflections. The output from the audio emotion classifier (2102) identifies speech emotions and transmits phonetic emotional analysis data (217) which comprises a single data stream with at least one speech emotion but commonly multiple vocal emotions.

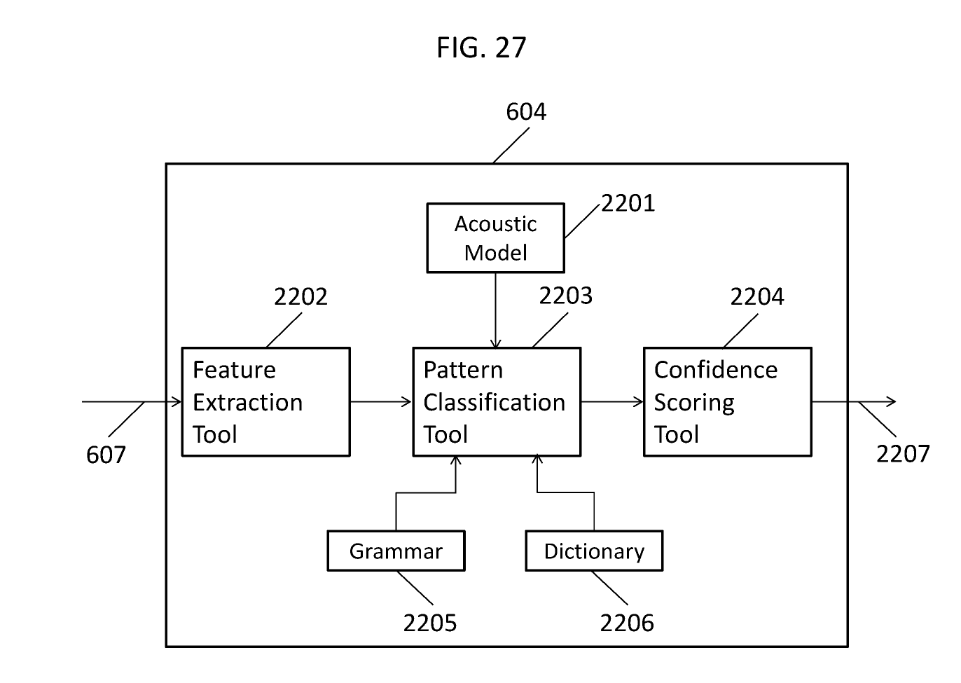

FIG. 27 depicts an embodiment of a speech recognition module (604), a component within an audio preprocessor (see FIG. 11). The speech recognition module (604) may have an acoustic model (2201), a feature extraction tool (2202), a pattern classification tool (2203), a confidence scoring tool (2204), a grammar module (2205), and a dictionary (2206). Speaker diarization output (607) is received by the feature extraction tool (2202) for processing. Vocal feature data is transmitted to a pattern classification tool (2203). Acoustic model data (2201), grammar data, and dictionary data are also sent to the pattern classification tool for processing with the vocal feature data. Pattern data is transmitted to the confidence scoring tool (2204) and speech recognition module output (2207), commonly in the form of text, for combination with other audio preprocessor output (not shown).

Alternate embodiments of the speech recognition module may include a machine learning architecture, where audio data (210) is received and transcribed audio is the output (2207). One embodiment includes a framework such as a recurrent neural network,

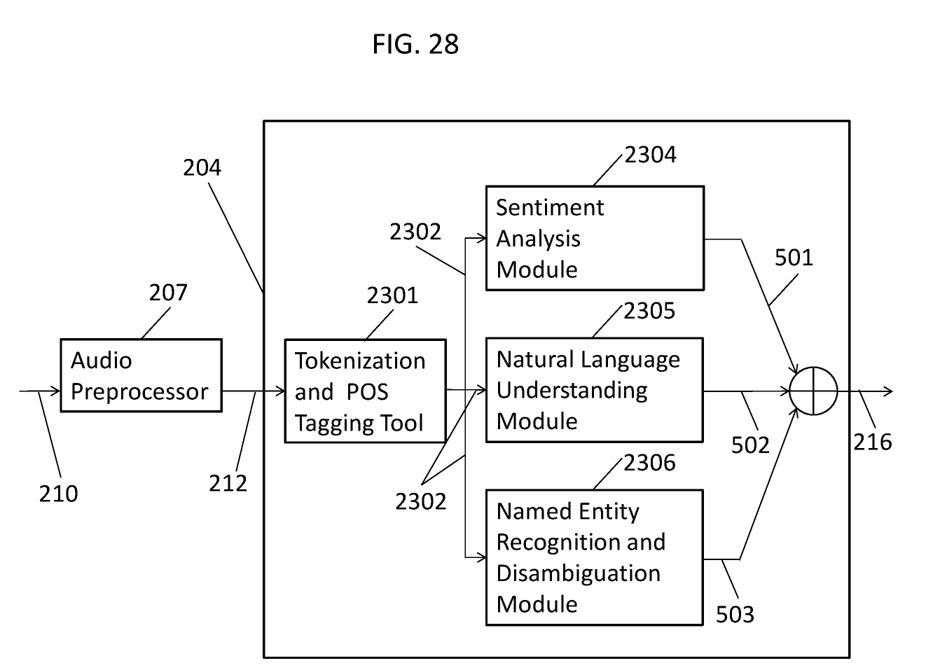

FIG. 28 depicts a natural language processing system (204). Shown are an audio preprocessor (207), a tokenization and part of speech (POS) tagging tool (2301), a sentiment analysis tool (2304), a natural language understanding module (2305), and a named entity recognition and disambiguation module (2306). Audio output (210) from a data input device (not shown) is received by the audio preprocessor (207), for processing, and audio preprocessor output is transmitted to the natural language processing system (204). The audio preprocessor output (212) is received by the tokenization and POS tagging tool (2301). The tokenization and POS tagging tool (2301) performs data processing and transmits tokenization and POS data (2302) to the sentiment analysis tool (2304), the natural language understanding tool (2305), and the named entity recognition tool (2306). The sentiment analysis tool (2304), processes tokenization and POS data (2302) and transmits sentiment data (501). The natural language understanding tool (2306) processes tokenization and POS data (2302) and transmits intent data (502). The named entity recognition tool (2306) processes tokenization and POS data (2302) and transmits entity recognition data (503). Sentiment data (501), intent data (502), and entity recognition data (503), which is depicted as separate streams but is often combined into a single data stream, natural language output data (216) for transmission. Sentiment data may be classified as positive, negative, or neutral. Intent data will vary with the application, but in a retail setting intent results may include but not be limited to factors such as: a willingness to buy because of price or because of quality, or a reluctance to buy because of price or brand. Entity recognition may vary with the application but in a retail setting, identified entities may include available merchandise, unavailable merchandise, and other stores or companies.

For natural language processing, speech recognition, or natural language processing systems, systems can be trained for any language or on multiple languages.

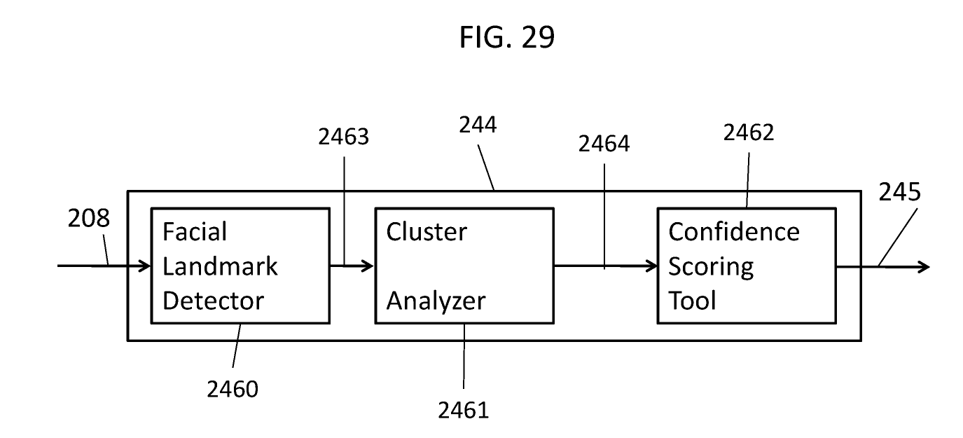

FIG. 29 depicts a facial recognition module (244). Shown are a facial landmark detector (2460), a cluster analyzer (2461), and a confidence scoring tool (2462). Video data (208) is transmitted to the facial recognition module (244). The video data (208) is received by the facial landmark detector (2460) for processing. Facial landmark data (2463) is transmitted to the cluster analyzer (2461) and is received by the cluster analyzer (2461) for processing. Facial landmark data (2463) may be in the form of data objects that characterize various elements of a face identified in the video data (208). The cluster analyzer (2461) transmits cluster analysis data (2464) to the confidence scoring tool (2462). The cluster analysis data (2464) is a set of similar images that bear close resemblance to each other and to the input facial landmark data (2463). The confidence scoring tool (2462) receives the cluster analysis data (2464) for processing. The confidence scoring tool (2462) identifies whether a matched image is found. The facial recognition module (244) may include matched image data in the transmitted facial recognition module output data (245).

The distributed system for building user profiles (100) collects input data about a subject from multiple data input devices (103). As a subject moves about a fixed space, the data input devices will collect and update data. In a retail setting, video, audio, spatial recognition data, and electronic device identification data may be collected and a large amount of information may be gathered on a person's retail shopping habits. The actual data collected for customer profiles will vary from retailer to retailer, making an assortment of emotional data, identity data, product data, and purchasing data available for market research. Some potential data items include but are not limited to: a subject's identity, visit frequency, purchase amount, merchandise preference, foot-traffic patterns, emotional response to products, emotional response to brands, emotional response to pricing, demographic analysis, connection with loyalty programs and program profiles, and connection with off-site persona data.

Visual items that may be part of a database include but are not limited to facial recognition, facial expression recognition, gaze-tracking, and demographic analysis data. Audio items that may be part of the database include but are not limited to phonetic emotional analysis and natural language processing, yielding sentiment data (501), intent data (502), and entity recognition data (503). Electronic device identification provides unique electronic device identification data and the spatial position module (107) provides position data both for the user and for the input device. The assortment of data items collected provide a way to correlate visual, sound, and emotional queues with store products the customer views, selects and/or ultimately purchases. The system may also allow for redundant checks to ensure data correctness by providing comparisons and corrections as a person moves through the store.

Data input devices (103) are positioned around a retail location. The position of a data input device (103) may be determined during setup by taking a picture of barcodes in the vicinity, or sensing RFID tags attached to merchandise, or by relative position in a network using Bluetooth® signals captured from BLE beacons or through a positioning method that uses the data input device's own network connection. The data input device (103) can also be calibrated, allowing the adjustment of the video input module (104) height and viewing angle. The employee interface device (1201) is used to set-up the data input device modules and to establish or update a planogram that resides in the at least one primary data repository (1103). The planogram provides location information that aides in product identification for gaze tracking. The employee interface device (1201) may also receive alarms from a data input device (103), as the employee interface device (1201) communicates with the data input device (103) and the profile building system (101). Alarms include but are not limited to tampering, low battery, no sound, no video, obstruction, displacement, and other matters which affect proper operation of the data input device (103).

The data input device (103) is not limited to a particular configuration, structure or type of input devices. It is not limited to a single camera or microphone, but may be a cluster, strip, or any configuration that allows for at least one video input module (104), at least one audio input module (105), at least one electronic device identification module (106), and at least one spatial position module (107).

The network of distributed data input devices (103), when triggered, send data to a behavior learning system (102), for processing, and then to a profile building system to build user profiles. As a subject walks within sensor range of a spatial position module (107), data gathering for that person's profile is triggered. Video, sound, subject spatial position data, and subject electronic device identification data are gathered. Audio and video input devices may be sufficiently sophisticated so that even in a group of people, a profile may be created and/or updated for each person in a group.

In some situations, video, audio, electronic device identification, or even spatial data may not be available. What data is received will be streamed to a behavior learning system. The system builds or updates a user profile with what data is available.

Video data (1004), audio data (1005), electronic device identification data (1006), and spatial position data (1007) is sent a behavior learning system. At least one data input device processor (108) may process, organize, coordinate, aggregate, separate, stream, direct, or control data flow.

The behavior learning system receives data input device output (1008). At least one behavior learning processor (109) may process, organize, coordinate, aggregate, separate, stream, direct, or control data flow. In an embodiment where the behavior learning system (102) is within a data input device (103), the behavior learning processor (109) and the data input processor (108) may be the same device. The behavior learning processor (109) may take a snapshot from the video data (208) feed and provides image output data (209) for data going to the at least one demographic analysis module (203). Within the behavior learning system, the video processor (110) receives video data (208), image data (209), and spatial position data, using one of the modules within the video processor (110) to processes the data. The audio processor (111) receives audio data (210) and uses one of the modules within the audio processor (111) to process the data.

At least one facial recognition module (244) performs face detection, face classification, and face recognition. The facial recognition module may provide facial recognition based on stored data in a one-to-many comparison, and/or a one-to-one comparison, and/or a one-to-few comparison. If there is a match, the output is sent in the form of facial recognition module output data (245).

At least one facial expression recognition module (202) analyzes expressions to determine a person's emotional reactions and the strength of the emotional reaction. The output is transmitted as facial expression recognition output data (213).

At least one gaze tracking module (201) determines a person's gaze direction, using planogram data (711) to identify products the users looks at. Often in the form of target merchandise data (710), gaze tracking data (214) is transmitted.

At least one demographic analysis module (203) determines the age (505), race (506), and gender (507) of a subject.

At least one audio preprocessor (207) receives audio data (210) and provides and speech recognition module output (2207) as audio preprocessor output (212). The audio preprocessor output (212) acts as input for at least one natural language processing module (204) and for at least one phonetic emotional analysis module (205).

The natural language processing module (204) provides sentiment data (501), intent data (502), and entity recognition data (503) commonly in relation to merchandise, when used in retail settings. However, natural language processing may be targeted for other market feedback, including but not limited to displays, layouts, staff, or other store features.

The emotional analysis module (205) provides output which identifies a subject's emotional reactions. Emotional reactions may vary as a person moves through a fixed space, or an item may trigger multiple emotional reactions, or a person may have varying intensities of a single emotion.

The entire system performs so that data input devices (103) are simultaneously collecting input data on multiple people within range of different data input devices within the fixed-space. The behavior learning system is simultaneously performing data analysis on multiple people, and multiple user profiles are simultaneously being built and/or updated. Face-recognition, facial expression recognition, gaze tracking, demographic analysis, speech recognition, and natural language processing, may be performed on group members within the field of view of a data input device (103) simultaneously and profiles can be created and/or updated on individual group members simultaneously. Not all modules need to collect data at the same time and there are times where certain data will be collected but other data will not. For example, if a subject is silent, then video data (1004), electronic device identification data (1006), and spatial position data (1007) will be collected and the profile updated.

Identification of a subject can be performed based on electronic device identification and/or facial recognition. If no video data (1004) is available a profile may be made using just electronic device identification. If the electronic device identification signal is not available or multiple signals are detected because a person is carrying multiple devices, a person's identity may be created and/or updated based solely on facial recognition. When both the electronic device and the face can be identified, it allows creation of an offsite persona. For the offsite persona, commonly collected data includes the MAC ID and IP address.

An electronic kiosk involves either direct interaction between the subject and an electronic device or between the subject and an intermediary person operating an electronic device, to complete a transaction, where the electronic device collects transactional information about the subject and the subject's interaction. The electronic device transmits electronic kiosk data, which is the transactional information. The electronic kiosk data is most commonly stored in the at least one primary data repository and may be used in building the user profile. Examples of electronic kiosks include but are not limited to point of sale terminals, airport boarding-pass dispensary machines, security checkpoints involving identification cards, security screening checkpoints, and such devices. Examples of transactions include but are not limited to service or product purchases, service or product confirmation document collection, electronic identification document scanning.

Purchasing data may also be significant. A common embodiment is to match the timestamp at the time items were purchased from a point of sale terminal with a timestamp of identity capture by the data input device (103) located near the point of sale terminal as the person is making a purchase. In this embodiment, items purchase can be associated with a person's identity. Since a data input device (103) receives video input (1040) and spatial position input (1070), another option is for the system to use the video input (1040) and spatial position input (1070) to determine what products the customer purchased and provide a timestamp. Another option is to collect purchase data through membership in a loyalty program that is commonly stored in either the primary data repository (1103) or in a secondary data repository (1104). A still further option is to track user purchases through RFID readers (403) that may be present on the data input device (103).

Subject identity is used to build the user profile. Subject identity is determined using a biometric identifier, and/or mobile electronic device identification data, and/or at least one establishment identifier. Biometric identifiers most commonly include facial recognition. However, other biometric identifiers may include but are not limited to voice recognition, gait recognition, or iris identification. Mobile electronic device identification data includes the MAC ID and/or the Bluetooth® mobile electronic device address data.

The profile may include mobile electronic device identification data for more than one mobile device. The at least one establishment identifier will depend on what the purpose of the fixed space is for and may depend on the establishment. In a retail setting, a loyalty card or “app” commonly provide the establishment identifier.

As a customer moves through a fixed space, data is gathered and periodically updated. The profile building system (101) may provide instructions to the employee interface device (1201). Such instructions may include directing an employee to assist a customer, or directing an employee to make special offers to the customer.

### Non-Limiting Embodiments

Embodiment 1 is a distributed system for building a plurality of user profiles comprising a distributed system for building a plurality of user profiles having a user profile from the plurality of user profiles having user profile data; at least one profile building system comprising at least one behavioral response analysis system and the plurality of user profiles; at least one behavior learning system comprising at least one behavior learning processor, at least one video data processor, and at least one audio data processor; at least one data input device having a data input device processor and/or at least one video input module, and/or at least one audio input module, and/or at least one electronic device identification module, and/or at least one spatial position module; and a data communication network comprising the at least one profile building system, the at least one behavior learning system, and the at least one data input device.

Embodiment 2 is the distributed system for building a user profile of embodiment 1, where the at least one video data processor has at least one gaze tracking module, and/or at least one facial expression recognition module, and/or at least one facial recognition module, and/or at least one demographic analysis module.

Embodiment 3 is the distributed system for building a user profile of embodiment 2, wherein the at least one audio data processor comprises at least one phonetic emotional analysis module, and/or at least one audio preprocessor module, and/or at least one natural language processing module.

Embodiment 4 is the distributed system for building a user profile of embodiment 3, where at least one behavioral response analysis system comprises at least one stream processing engine, at least one analytics engine, and at least one primary data repository; wherein the plurality of user profiles are stored in the at least one primary data repository.

Embodiment 5 is the distributed system for building a user profile of embodiment 4, where the at least one profile building system further comprises an administration module and at least one secondary data repository.

Embodiment 6 is the distributed system for building a user profile of embodiment 3, where the at least one behavior learning system is a component of the at least one data input device, and/or an independent system, and/or the at least one profile building system.

Embodiment 7 is the distributed system for building a user profile of embodiment 1, wherein the at least one electronic device identification module is a Wi-Fi packet analyzer module, and/or a mobile device Bluetooth® identification module.

Embodiment 8 is the distributed system for building a user profile of embodiment 1, where the at least one spatial position module comprises a range finder sensor, and a spatial data gathering device selected from a barcode reader, and/or an RFID reader, and/or a Bluetooth® Low Energy receiver, and/or a Wi-Fi positioning module.

Embodiment 9 is the distributed system for building a user profile of embodiment 1, where the data communication network is connected to at least one employee interface device.

Embodiment 10 is the at least one video data processor of embodiment 2, where the at least one video data processor comprises a gaze tracking module and the gaze tracking module comprises a computer vision system, a transfer function module, and an attribution module.

Embodiment 11 is a distributed system for building a plurality of user profiles comprising: a distributed system for building a plurality of user profiles having, a user profile from the plurality of user profiles having user profile data; at least one profile building system building the user profile comprising at least one behavioral response analysis system providing behavioral response analysis data, and the plurality of user profiles; at least one behavior learning system comprising at least one behavior learning processor, at least one video data processor providing video processor data, and at least one audio data processor providing audio processor data; at least one data input device comprising a data input device processor and data input modules providing data from at least one video input module providing video data, and/or at least one audio input module providing audio data, and/or at least one electronic device identification module providing electronic device identification data, and/or at least one spatial position module providing spatial position data; and a data communication network providing data communication comprising the profile building system, the behavior learning system, and the at least one data input device.

Embodiment 12 is the distributed system for building a user profile of embodiment 11, where the at least one video data processor providing video processor data from at least one gaze tracking module providing gaze tracking data, and/or at least one facial expression recognition module providing facial expression recognition data, and/or at least one facial recognition module providing facial recognition data, and/or at least one demographic analysis module providing demographic analysis data.

Embodiment 13 is the distributed system for building a user profile of embodiment 12, where the at least one audio data processor providing audio processor data comprises audio processor data from at least one phonetic emotional analysis module providing phonetic emotional analysis data, and/or at least one audio preprocessor module providing audio preprocessor data, and/or at least one natural language processing module providing natural language processing data.

Embodiment 14 is the distributed system for building a user profile of embodiment 13, where at least one behavioral response analysis system providing behavioral response analysis data comprising at least one stream processing engine, at least one analytics engine, and at least one primary data repository; wherein the plurality of user profiles are stored in the at least one primary data repository.

Embodiment 15 is the at least one profile building system of embodiment 14, where the at least one profile building system building the user profile comprising user profile data receives from at least one gaze tracking module providing gaze tracking data, and/or at least one facial expression recognition module providing facial expression recognition data, and/or at least one facial recognition module providing facial recognition data, and/or at least one demographic analysis module providing demographic analysis data, and/or at least one phonetic emotional analysis module providing phonetic emotional analysis data, and/or at least one audio preprocessor module providing audio preprocessor data, and/or at least one natural language processing module providing natural language processing data, and/or at least one spatial position module providing spatial position data, and/or at least one electronic device identification module providing electronic device identification data, and/or at least one behavioral response analysis system providing behavioral response analysis data comprising.

Embodiment 16 is the distributed system for building a user profile of embodiment 15, where the at least one profile building system further comprises an administration module and at least one secondary data repository providing secondary data; and where the user profile from the plurality of user profiles further comprises secondary data.

Embodiment 17 is the distributed system for building a user profile of embodiment 11, where the at least one behavior learning system further is a component from at least one data input device, and/or an independent system, and/or the at least one profile building system.

Embodiment 18 is the distributed system for building a user profile of embodiment 11, where the at least one electronic device identification module providing electronic device identification data is a Wi-Fi packet analyzer module providing Wi-Fi packet analysis data, and/or a mobile device Bluetooth® identification module providing mobile device Bluetooth® identification data.

Embodiment 19 is the distributed system for building a user profile of embodiment 11, where the at least one spatial position module providing spatial position data; where the spatial position data comprises absolute position data, relative position data, height data, and horizontal distance data; and where the spatial position data is selected from a barcode reader providing barcode data, and/or a range finder sensor providing range data, and/or an RFID reader providing RFID data, and/or a Bluetooth® Low Energy receiver providing Bluetooth® Low energy data, and/or a Wi-Fi positioning module providing Wi-Fi positioning data.

Embodiment 20 is the at least one video data processor of embodiment 12, where the at least one video data processor providing video processor data comprises a gaze tracking module providing gaze tracking data; where the gaze tracking module providing gaze tracking data comprises a computer vision system providing video gaze output data, a transfer function module providing field-of-view data, and an attribution module providing target merchandise data; and where gaze tracking data comprises target merchandise data.

Embodiment 21 is the distributed system for building a user profile of embodiment 16, where demographic analysis data comprises race data, age data, and gender data.

Embodiment 22 is the distributed system for building a user profile of embodiment 16, where the administration module comprises a dashboard and administrative tools.

Embodiment 23 is the distributed system for building a user profile of embodiment 11, where the data communication network providing data communication further comprises at least one employee interface device receiving employee instructions, data input device alarms, and data input device provisioning instructions.

Embodiment 24 is a method for building a user profile, the method steps comprising: providing at least one data input device of a plurality of data input devices in at least one fixed space collecting and transmitting video data, audio data, mobile electronic device identification data, and spatial position data of a person from a plurality of persons as the person moves throughout the at least one fixed space; at least one behavior learning system receiving video data, audio data, mobile electronic device identification data, and spatial position data, having at least one video data processor processing video data and at least one audio data processor processing audio data; the at least one behavior learning system transmitting mobile electronic device identification data, spatial position data, video processor data and audio processor data; at least one profile building system receiving mobile electronic device identification data, spatial position data, video processor data, and audio processor data, and building the user profile of the plurality of user profiles; where the plurality of user profiles are stored in at least one primary data repository.

Embodiment 25 is the method of embodiment 24, wherein the at least one video data processor comprises: at least one gaze tracking module performing gaze tracking analysis and transmitting gaze tracking data, at least one facial recognition module performing facial recognition analysis and transmitting facial recognition data, at least one facial expression recognition module performing facial expression recognition analysis and transmitting facial expression recognition data, at least one demographic analysis module performing demographic analysis and transmitting demographic analysis data, and wherein video processor data comprises gaze tracking data, facial recognition data, facial expression recognition data, and demographic analysis data.

Embodiment 26 is the method of embodiment 25 wherein the at least one audio data processor comprises: at least one audio preprocessor module performs audio preprocessor analysis, and transmits audio preprocessor data; at least one phonetic emotional analysis module receiving audio preprocessor data, performing phonetic emotional analysis and transmitting phonetic emotional analysis data; at least one natural language processing module receiving audio preprocessor data, performing natural language understanding, performing sentiment analysis, and performing named entity recognition, and transmitting natural language processing data comprising natural language understanding data, sentiment analysis data and named entity recognition data; and wherein the audio processor data comprises phonetic emotional analysis data and natural language processing data.

Embodiment 27 is the method of embodiment 26, wherein the profile building system further comprises: associating the user profile from the plurality of user profiles with secondary data selected from at least one secondary data repository; the at least one behavioral response analysis system performing analysis of user profile data and secondary data; and updating the user profile.

Embodiment 28 is the method of embodiment 27, wherein the profile building system transmits instructions to at least one employee interface device, where the employee interface device receives instructions, and communicates said instructions to an employee through an employee application computer program.

Embodiment 29 is the method of embodiment 24 wherein the profile building system further comprises: the at least one behavioral response analysis system receiving video data, electronic device identification data, and spatial position data to create traffic data selected from the group consisting of a heat map, queue analysis data, traffic analysis data, people count data, and combinations thereof, and where the primary data repository stores retail data.

Embodiment 30 is the method of embodiment 25, where the gaze tracking module receives video data and spatial position data, where a computer vision system determines eye position and head orientation from the video data, transmitting eye position and head orientation data to a transfer function module; where the transfer function module receives eye position, head orientation data, and spatial position data; where input device field-of-view data, horizontal distance data, and height data are taken from the spatial data; where the transfer function module calculates user field of view data, and transmits the user field of view data to an attribution module, where the attribution module requests and receives planogram data from at least one primary data repository and receives the user field of view data, performing merchandise analysis, and transmitting gaze tracking data; and where gaze tracking data comprises target merchandize data.

Embodiment 31 is the method of embodiment 27, wherein the person interacts with an electronic kiosk providing electronic kiosk data, wherein at least one data input device collects and transmits video data, audio data, mobile electronic device identification data, and spatial position data of the person interacting with the electronic kiosk; wherein electronic kiosk data is transmitted to the primary data repository and/or the secondary data repository; and wherein the user profile further comprises electronic kiosk data.

Embodiment 32 is the method embodiment 31, where the electronic kiosk has a point of sale terminal, and wherein electronic kiosk data comprises product purchase data.

Embodiment 33 is the method of embodiment 32 wherein the product purchase data has a product identifier, sale amount, and a sale timestamp; wherein the profile building system provides a presence timestamp, location data, and identity data; wherein the sale timestamp and the presence timestamp are compared, user identity is confirmed, and stored sales data are selected from the product identifier, identity data, sale amount, sale timestamp, presence timestamp, location data, identity data, and combinations thereof.

Embodiment 34 is the method of embodiment 27 wherein the user profile from the plurality of user profiles is built using user identity, where user identity is at least one biometric identifier, and/or mobile electronic device identification data, and/or an establishment identifier.

Embodiment 35 is any one of embodiments 1-34 combined with any one or more embodiments 2-34.

What is claimed is:

1. A distributed system for building a plurality of user profiles comprising:
a distributed system for building a plurality of user profiles comprising,
a user profile from the plurality of user profiles comprising user profile data;
at least one profile building system comprising at least one behavioral response analysis system and the plurality of user profiles;
at least one behavior learning system comprising at least one behavior learning processor,
at least one video data processor, and at least one audio data processor;
at least one data input device comprising a data input device processor and an input data module selected from the group consisting of at least one video input module, at least one audio input module, at least one electronic device identification module, at least one spatial position module, and combinations thereof;
and a data communication network comprising the at least one profile building system,
the at least one behavior learning system, and the at least one data input device.

2. The distributed system for building a user profile of claim 1, wherein
the at least one video data processor comprises a video data processor module selected from the group consisting of at least one gaze tracking module, at least one facial expression recognition module, at least one facial recognition module, at least one demographic analysis module, and combinations thereof.

3. The distributed system for building a user profile of claim 2, wherein
the at least one audio data processor comprises an audio data processor module selected from the group consisting of, at least one phonetic emotional analysis module, at least one audio preprocessor module, at least one natural language processing module, and combinations thereof.

4. The distributed system for building a user profile of claim 3, wherein
at least one behavioral response analysis system comprises
at least one stream processing engine, at least one analytics engine, and at least one primary data repository; wherein
the plurality of user profiles are stored in the at least one primary data repository.

5. The distributed system for building a user profile of claim 4, wherein
the at least one profile building system further comprises:
an administration module and at least one secondary data repository.

6. The distributed system for building a user profile of claim 3, wherein
the at least one behavior learning system further is a component selected from the group consisting of the at least one data input device, an independent system, the at least one profile building system, and combinations thereof.

7. The distributed system for building a user profile of claim 1, wherein
the at least one electronic device identification module is selected from the group consisting of a Wi-Fi packet analyzer module, a mobile device Bluetooth® identification module, and combinations thereof.

8. The distributed system for building a user profile of claim 1, wherein
the at least one spatial position module comprises a range finder sensor, and a spatial data gathering device selected from the group consisting of a barcode reader, an RFID reader, a Bluetooth® Low Energy receiver, a Wi-Fi positioning module, and combinations thereof.

9. The distributed system for building a user profile of claim 1, wherein
the data communication network further comprises at least one employee interface device.

10. The at least one video data processor of claim 2, wherein
the at least one video data processor comprises a gaze tracking module; wherein
the gaze tracking module comprises
a computer vision system, a transfer function module, and an attribution module.

11. A distributed system for building a plurality of user profiles comprising:
a distributed system for building a plurality of user profiles comprising,
a user profile from the plurality of user profiles comprising user profile data;
at least one profile building system building the user profile comprising at least one behavioral response analysis system providing behavioral response analysis data, and the plurality of user profiles;
at least one behavior learning system comprising at least one behavior learning processor,
at least one video data processor providing video processor data, and at least one audio data processor providing audio processor data;
at least one data input device comprising a data input device processor and data input modules providing data selected from the group consisting of at least one video input module providing video data, at least one audio input module providing audio data, at least one electronic device identification module providing electronic device identification data, at least one spatial position module providing spatial position data, and combinations thereof;
and a data communication network providing data communication comprising the profile building system, the behavior learning system, and the at least one data input device.

12. The distributed system for building a user profile of claim 11, wherein
the at least one video data processor providing video processor data comprises video processor data selected from the group consisting of at least one gaze tracking module providing gaze tracking data, at least one facial expression recognition module providing facial expression recognition data, at least one facial recognition module providing facial recognition data, at least one demographic analysis module providing demographic analysis data, and combinations thereof.

13. The distributed system for building a user profile of claim 12, wherein
the at least one audio data processor providing audio processor data comprises audio processor data selected from the group consisting of, at least one phonetic emotional analysis module providing phonetic emotional analysis data, at least one audio preprocessor module providing audio preprocessor data, at least one natural language processing module providing natural language processing data, and combinations thereof.

14. The distributed system for building a user profile of claim 13, wherein
at least one behavioral response analysis system providing behavioral response analysis data comprising
at least one stream processing engine, at least one analytics engine, and at least one primary data repository; wherein
the plurality of user profiles are stored in the at least one primary data repository.

15. The at least one profile building system of claim 14, wherein
the at least one profile building system building the user profile comprising user profile data received from the group consisting of at least one gaze tracking module providing gaze tracking data, at least one facial expression recognition module providing facial expression recognition data, at least one facial recognition module providing facial recognition data, at least one demographic analysis module providing demographic analysis data, at least one phonetic emotional analysis module providing phonetic emotional analysis data, at least one audio preprocessor module providing audio preprocessor data, at least one natural language processing module providing natural language processing data, at least one spatial position module providing spatial position data, at least one electronic device identification module providing electronic device identification data, at least one behavioral response analysis system providing behavioral response analysis data comprising, and combinations thereof.

16. The distributed system for building a user profile of claim 15, wherein
the at least one profile building system further comprises:
an administration module and at least one secondary data repository providing secondary data; and wherein
the user profile from the plurality of user profiles further comprises secondary data.

17. The distributed system for building a user profile of claim 11, wherein
the at least one behavior learning system further is a component selected from the group consisting of the at least one data input device, an independent system, the at least one profile building system, and combinations thereof.

18. The distributed system for building a user profile of claim 11, wherein
the at least one electronic device identification module providing electronic device identification data is selected from the group consisting of a Wi-Fi packet analyzer module providing Wi-Fi packet analysis data, a mobile device Bluetooth® identification module providing mobile device Bluetooth® identification data, and combinations thereof.

19. The distributed system for building a user profile of claim 11, wherein
the at least one spatial position module providing spatial position data; wherein
the spatial position data comprises absolute positions data, relative position data, height data, and horizontal distance data; and wherein
the spatial position data is selected from the group consisting of a barcode reader providing barcode data, a range finder sensor providing range data, an RFID reader providing RFID data, a Bluetooth® Low Energy receiver providing Bluetooth® Low energy data, a Wi-Fi positioning module providing Wi-Fi positioning data, and combinations thereof.

20. The at least one video data processor of claim 12, wherein
the at least one video data processor providing video processor data comprises a gaze tracking module providing gaze tracking data; wherein
the gaze tracking module providing gaze tracking data comprises
a computer vision system providing video gaze output data, a transfer function module providing field-of-view data, and an attribution module providing target merchandise data; and wherein gaze tracking data comprises target merchandise data.

21. The distributed system for building a user profile of claim 16, wherein demographic analysis data comprises race data, age data, and gender data.

22. The distributed system for building a user profile of claim 16, wherein
the administration module comprises a dashboard and administrative tools.

23. The distributed system for building a user profile of claim 11, wherein
the data communication network providing data communication further comprises at least one employee interface device receiving employee instructions, data input device alarms, and data input device provisioning instructions.

24. A method for building a user profile, the method steps comprising:
providing at least one data input device of a plurality of data input devices in at least one fixed space collecting and transmitting video data, audio data, mobile electronic device identification data, and spatial position data of a person from a plurality of persons as the person moves throughout the at least one fixed space;
at least one behavior learning system receiving video data, audio data, mobile electronic device identification data, and spatial position data, having at least one video data processor processing video data and at least one audio data processor processing audio data; the at least one behavior learning system transmitting mobile electronic device identification data, spatial position data, video processor data and audio processor data;
at least one profile building system receiving mobile electronic device identification data, spatial position data, video processor data, and audio processor data, and building a user profile of the plurality of user profiles; wherein
the plurality of user profiles are stored in at least one primary data repository; and
wherein
the user profile is updated for each person from the plurality of persons moving throughout the at least one fixed space.

25. The method of claim 24, wherein the at least one video data processor comprises:
at least one gaze tracking module performing gaze tracking analysis and transmitting gaze tracking data;
at least one facial recognition module performing facial recognition analysis and transmitting facial recognition data;
at least one facial expression recognition module performing facial expression recognition analysis and transmitting facial expression recognition data;
at least one demographic analysis module performing demographic analysis and transmitting demographic analysis data;
and wherein video processor data comprises gaze tracking data, facial recognition data, facial expression recognition data, and demographic analysis data.

26. The method of claim 25 wherein the at least one audio data processor comprises
at least one audio preprocessor module performs audio preprocessor analysis, and transmits audio preprocessor data;
at least one phonetic emotional analysis module receiving audio preprocessor data, performing phonetic emotional analysis and transmitting phonetic emotional analysis data;
at least one natural language processing module receiving audio preprocessor data, performing natural language understanding, performing sentiment analysis, and
performing named entity recognition, and transmitting natural language processing data comprising natural language understanding data, sentiment analysis data and named entity recognition data; and wherein
audio processor data comprises phonetic emotional analysis data and natural language processing data.

27. The method of claim 26, wherein the profile building system further comprises:
associating the user profile from the plurality of user profiles with secondary data selected from at least one secondary data repository;
the at least one behavioral response analysis system performing analysis of user profile data and secondary data;
and updating the user profile.

28. The method of claim 27, wherein the profile building system transmits instructions to at least one employee interface device, wherein
the employee interface device receives instructions, and communicates said instructions to an employee through an employee application computer program.

29. The method of claim 24 wherein the profile building system further comprises:
the at least one behavioral response analysis system receiving video data, electronic device identification data, and spatial position data to create traffic data selected from the group consisting of a heat map, queue analysis data, traffic analysis data, people count data, and combinations thereof; and wherein
the primary data repository stores traffic data.

30. The method of claim 25, wherein
the gaze tracking module receives video data and spatial position data, wherein
a computer vision system determines eye position and head orientation from the video data, transmitting eye position and head orientation data to a transfer function module;
wherein
the transfer function module receives eye position, head orientation data, and spatial position data; wherein
input device field-of-view data, horizontal distance data, and height data are taken from the spatial data; wherein
the transfer function module calculates user field of view data, and transmits the user field of view data to an attribution module, wherein
the attribution module requests and receives planogram data from at least one primary data repository and receives the user field of view data, performing merchandise analysis, and transmitting gaze tracking data; and wherein
gaze tracking data comprises target merchandise data.

31. The method of claim 27, wherein
the person interacts with an electronic kiosk providing electronic kiosk data, wherein at least one data input device collects and transmits video data, audio data, mobile electronic device identification data, and spatial position data of the person interacting with the electronic kiosk; wherein electronic kiosk data is transmitted to data storage selected from the group consisting of the primary data repository, the secondary data repository, and combinations thereof, and wherein
the user profile further comprises electronic kiosk data.

32. The method of claim 31, wherein
the electronic kiosk comprises a point of sale terminal, and wherein
electronic kiosk data comprises product purchase data.

33. The method of claim 32, wherein
the product purchase data comprises a product identifier, sale amount, and a sale timestamp; wherein
the profile building system provides a presence timestamp, location data, and identity data, wherein
the sale timestamp and the presence timestamp are compared, user identity is confirmed, and stored sales data are selected from the product identifier, identity data, sale amount, sale timestamp, presence timestamp, location data, identity data, and combinations thereof.

34. The method of claim 27, wherein
the user profile from the plurality of user profiles is built using user identity, wherein user identity is selected from the group of at least one biometric identifier, mobile electronic device identification data, an establishment identifier, and combinations thereof.
